(19) United States
(12) Patent Application Publication
(10) Pub. No.: US 2019/0241982 A1
(43) Pub. Date: Aug. 8, 2019

(54) SYSTEMS AND METHODS FOR TRACKING THE ORIGIN OF CANNABIS PRODUCTS AND CANNABIS DERIVATIVE PRODUCTS

(71) Applicant: APDN (B.V.I.) Inc., Tortola (VG)

(72) Inventors: Michael E. HOGAN, Stony Brook, NY (US); Gordon HOPE, Stony Brook, NY (US); James A. HAYWARD, Stony Brook, NY (US); John SHERMAN, Smithtown, NY (US); Thomas ACKERMAN, Farmingville, NY (US); Brian VISCOUNT, Mt. Sinai, NY (US)

(73) Assignee: APDN (B.V.I.) Inc., Tortola (VG)

(21) Appl. No.: 16/265,631

(22) Filed: Feb. 1, 2019

Related U.S. Application Data
(60) Provisional application No. 62/625,702, filed on Feb. 2, 2018.
(60) Provisional application No. 62/700,021, filed on Jul. 18, 2018.

(51) Int. Cl. C12Q 1/6895

(52) U.S. Cl. CPC C12Q 1/6895

(57) ABSTRACT

The invention provides for a system to track the origin of cannabis products and cannabis derivative products without the need for packaging or labeling through the use of nucleic acid tags. The invention further provides for a method of tracking the origin of cannabis products and cannabis derivative products via the application of nucleic acid tags to cannabis plants.

### CROSS-REFERENCE TO RELATED APPLICATION

This application claims the benefit of U.S. Provisional Patent Application No. 62/625,702, filed on Feb. 2, 2018, and U.S. Provisional Patent Application No. 62/700,021, filed on Jul. 18, 2018, both of which are hereby incorporated by reference in their entireties.

### TECHNICAL FIELD

The invention provides for a system to track the origin of cannabis products and cannabis derivative products without the need for packaging or labeling through the use of nucleic acid tags. The invention further provides for a method of tracking the origin of cannabis products and cannabis derivative products via the application of nucleic acid tags to cannabis plant material.

### BACKGROUND OF THE INVENTION

The legal cannabis market is rapidly expanding. This expansion has created a need for a system and method capable of ascertaining the provenance of cannabis products and/or cannabis derivative products without relying on easily circumvented packaging and labels. In addition, with the expansion of legally permitted cannabis growing operations, the illegal diversion of legally grown cannabis into other jurisdictions where cannabis is illegal is an ongoing and unsolved problem.

Current cannabis “seed-to-sale” systems, which rely on barcodes, RFID technologies and packaging/labeling materials, are capable of tracking a cannabis supply chain through cultivation, but cannot track the provenance of the final cannabis products and/or cannabis derivative products. This is especially true if the cannabis products and/or cannabis derivative products are dissociated from their packaging as, under current systems, all indicia of origin reside on the product packaging or labeling. Thus, under current systems, once cannabis products and/or cannabis derivative products are removed from their packaging, there is no way to determine the origin of the product. This fact allows illegal cannabis operations to supply cannabis into legal markets and for legal cannabis to be diverted to non-permissive jurisdictions. The implementation of a system and/or method wherein the actual cannabis product and cannabis derivative product is indelibly tagged with a nucleic acid tag is the only way to address these ongoing, and yet unsolved problems.

### SUMMARY OF THE INVENTION

A system for tracking the origin of cannabis products and cannabis derivative products is disclosed. The system comprises: (a) a nucleic acid tag; (b) a marking apparatus; (c) a nucleic acid tag authentication apparatus, and optionally; (d) a digital data repository wherein the marking apparatus and the nucleic acid tag authentication apparatus are configured to digitally communicate with the digital data repository system; and (e) one or more remote user interfaces to the digital data repository.

In certain embodiments the nucleic acid tag is a DNA tag, comprised of one or more known DNA sequences. The DNA tag may contain one or more discrete informational units. The nucleic acid tag may also be a combinatorial nucleic acid mark comprised of multiple DNA fragments, each fragment having a different known DNA sequence. The nucleic acid tag may be a combinatorial DNA tag comprised of one or more an amplicons and one or more oligonucleotides. The nucleic acid tag may be created on demand via an oligonucleotide synthesis apparatus, which may be configured to digitally communicate with the digital data repository system and/or the marking apparatus. The marking apparatus may be comprised of a misting system, electrostatic spray system, atomized spray system, pressurized spray system, sprinkler system, fogging system, cooling system, handheld spray device, powder dusting system, or any combination thereof. The authentication apparatus may be comprised of a capillary electrophoresis apparatus, qPCR apparatus, PCR apparatus, microarray, microarray scanner, multi-mode reader, next generation sequencing apparatus, Sanger sequencing apparatus, Luminex or other multiplexing system, or any combination thereof.

A method of tracking the origin of cannabis products and cannabis derivative products is also disclosed. The method comprises: (a) applying a nucleic acid tag to cannabis plant material to create nucleic acid tagged cannabis plant material, said tag imparting at least one piece of information about the origin of the cannabis plant material; (b) processing the nucleic acid tagged cannabis plant material to create nucleic acid tagged cannabis products and/or nucleic acid tagged cannabis derivative products; (c) interrogating a nucleic acid tagged cannabis product and/or cannabis derivative product to ascertain information from the nucleic acid tag; and optionally; (d) querying a digital data repository for information associated with the nucleic acid tag information; and (e) utilizing the nucleic acid tag information in conjunction with the information form the digital data repository to ascertain the source of the cannabis product and/or cannabis derivative product.

In certain embodiments the nucleic acid tag is a DNA tag, comprised of one or more known DNA sequences. The nucleic acid tag may also be a combinatorial nucleic acid mark comprised of multiple DNA sequences, each having a different known sequence. The nucleic acid tag may be a DNA tag comprised of combination of amplicons and oligonucleotides. The nucleic acid tag may be created on demand via an oligonucleotide synthesis apparatus, or oligonucleotides may be combined with pre-manufactures amplicons to create on bespoke DNA tags. The DNA tag may be applied to a growing or cultivated cannabis plant via a misting system, electrostatic spray system, atomized spray system, sprinkler system, pressurized spray system, fogging system, cooling system handheld spray device, powder dusting system, or any combination thereof. The interrogation of the cannabis products and/or cannabis derivative products may be performed via a capillary electrophoresis apparatus, qPCR apparatus, PCR apparatus, microarray, microarray scanner, next generation sequencing apparatus, Sanger sequencing apparatus, nanopore sequencing apparatus, multiplex qPCR apparatus, multi-mode reader or Luminex or other multiplexing system, or any combination thereof.

In one embodiment, a system of applying a taggant to plant material is provided. The system comprises: a spraying chamber including panels defining an interior for receiving plant material; an attachment assembly adapted to secure the plant material within the spraying chamber; a fogging unit in fluid communication with the spraying chamber; an aqueous supply of taggant solution in fluid communication with the fogging unit, the fogging unit producing a mist from the solution without the use of heat; a mist dispersal device disposed adjacent the fogging unit for disbursing mist produced by the fogging unit to coat the plant material with the nucleic acid tag solution (“nucleic acid marker solution”); and a processing device for controlling the operations of the fogging unit and mist dispersal device.

### BRIEF DESCRIPTION OF THE FIGURES

FIG. 1 is a flow diagram of one embodiment of the disclosed system.

FIG. 2 is a flow diagram of an alternative embodiment of the disclosed system.

FIG. 3 is a flow diagram of one embodiment of the disclosed method.

FIG. 4 illustrates a block diagram of a general computer system that can perform any one or more of the methods or computer based functions disclosed herein.

FIG. 5A illustrates one embodiment of a DNA tag comprised of an amplicon with one or more discrete informational units.

FIG. 5B illustrates an embodiment of a combinatorial DNA tag comprised of an amplicon and an oligonucleotide, each with one or more discrete informational units.

FIG. 6A and 6B illustrates exemplary combinatorial DNA tags comprised of an amplicon and an oligonucleotide, each with one or more discrete informational units.

FIG. 7 is schematic of the fogging (“spraying”) system of the present disclosure.

FIG. 8 is a perspective view of the fogging system of the present disclosure.

FIG. 9 is an elevational front view of the fogging system.

FIG. 10 is a side elevational view of the fogging system.

FIG. 11 is a perspective view of the fogging system solution storage.

FIGS. 12-13 are descriptions of processing devices of the fogging system.

FIG. 14 is a screen flow of a touchscreen display.

FIGS. 15-20 are screen displays of the touchscreen display.

FIG. 21 is a flow chart of one exemplary embodiment of the fogging system.

FIG. 21A presents data results of the fogging system

FIG. 22 a recitation of process steps during a fogging session showing the functions of the processing devices.

FIG. 23 is a recitation of steps showing communication between the fogging system processor and a third party verification system.

FIG. 24 is a data flow chart of a chain of custody for a molecular taggant.

FIG. 25 is a data flow chart of a DNA marking session.

FIG. 26 is a schematic of a fogging system integration to a third party verification system.

FIGS. 27 and 28 are depictions of the fogging system of the present disclosure.

FIG. 29 are side elevational, rear elevational and sectional views of one exemplary embodiment of the fogging system.

### DETAILED DESCRIPTION OF THE INVENTION

Definitions of terms used herein:

The term “amplicon” as used herein means a piece of linear DNA that is the product of an enzymatic or chemical-based amplification event or reaction. An amplicon may be single or double stranded. Amplification events or reactions include, without limitation, the polymerase chain reaction (PCR), loop mediated isothermal amplification, rolling circle amplification, nucleic acid sequence base amplification, and ligase chain reaction or recombinase polymerase amplification.

A “nucleic acid tag” (“nucleic acid taggant”) as used herein means any nucleic acid including at least in part, a unique sequence detectable by any of the many well known DNA detection techniques, including polymerase chain reaction (PCR) and microarray techniques, multiplexing techniques, qPCR techniques, as well as other methods of DNA amplification and detections such as isothermal, hybridization techniques and any of the well known method of DNA sequencing and sequence specific detection. A nucleic acid tag may be double or single stranded. A nucleic acid tag may be a DNA tag.

A “cannabis product” is any product comprised of dried portions of the cannabis plant that has not gone through an extraction or concentration, including but not limited to cannabis crown. Cannabis product also includes hemp and any member of the cannabis indicia and/or cannabis sativa family.

A “cannabis derivative product” is any product containing derivatives or concentrates from a cannabis or hemp plant created through chemical extraction, concentration or other methods. Examples of cannabis derivative products include, but are not limited to, products containing cannabidiol (CBD), CBD oils, extracted THC, THC oil, cannabis oil, cannabis hemp oil, Rick Simpson Oil, cannabis extract, cannabis concentrate, shatter (or other butane extract), and cannabis wax extracts.

### System for Tracking the Origin of Cannabis Products and Cannabis Derivative Products

In one aspect of the present invention, a system for the tracking of the origin of cannabis products and cannabis derivative products through the application, and subsequent analysis, of indelible nucleic acid tags applied to cannabis plant material is provided. In one embodiment, the system comprises: (a) one or more nucleic acid tags; (b) a marking apparatus; and (c) a nucleic acid tag authentication apparatus. In some embodiments, the system further comprises: (d) a digital data repository wherein the marking apparatus and the nucleic acid tag authentication apparatus are configured to digitally communicate with the digital data repository system; and (e) one or more remote user interfaces to the digital data repository.

### Nucleic Acid Tag

The one or more nucleic acid tags may be comprised of any suitable nucleic acid tag, including a DNA tag. The DNA may be single or double stranded DNA and of a known sequence and length. For example, the sequence is known to the provider of a tag. The DNA may be naturally occurring or synthetic. The DNA tag may be a single sequence from about 20 bases to about 5 Kb in single strand length, or about 20 base pairs to about 5 Kb pairs in double strand length. In an exemplary embodiment, the DNA is double stranded DNA of a known single sequence under 500 base pairs in length. The DNA tag may also be a combinatorial DNA tag comprised of one or more DNA fragments of known sequences, said DNA fragments each under 250 base pair in length.

The DNA tag may include an excess of carrier nucleic acids of a natural genomic sequence or a mixture of random synthetic or natural nucleic acid sequences. In this way, extraction of total nucleic acid will not reveal the detectable marker nucleic acid sequence without access to the cognate PCR primer pair or pairs for PCR, or the complementary nucleotide hybridization probe, depending on the detection method used. In one embodiment, the DNA tag may be comprised of a mixture of DNA and an ionic or non-ionic food grade surfactant. The DNA tag may also include a food grade or non-food grade encrypted or non-encrypted optical marker as disclosed in U.S. Patent Application 2015/0266332 A1, Szczepanik et al.

The DNA tag may also be in non-aqueous dry powdered form or be in an aqueous solution. In an exemplary embodiment, the dry powder DNA tag is comprised of a detectable nucleic acid marker and carrier complex created via a freeze drying (lyophilisation or the like) process. The carrier may be any suitable freeze dry carrier known in the art. The DNA tag may also be alkaline activated to increase binding affinity as disclosed in U.S. Pat. No. 9,790,538 to Berrada et al. and U.S. Pat. No. 9,266,370 to Jung et al. The DNA tag may include a combination of various non-ionic emulsifiers and/or surfactants. Exemplary compounds include Span 85, sorbitan trioelate, Tween 20 and Polysorbate 20, alkyl aryl polyoxyethylene glycol, Surfadone LP-100, Surfadone LP-300 and the like; polar aprotic solvents such as Acetone, DMSO, DMPU, 2-(2-Ethoxyethoxy) ethanol and the like. The DNA tag may also include chelating agents such as ethylenediaminetetraacetic acid (EDTA).

The DNA tag may comprise at least one primer binding site and one or more discrete informational units. Each discrete informational unit may be configured to impart a specific piece of data upon authentication of the DNA tag. In an embodiment, the DNA tag comprises one or more known synthetic DNA sequences, each under 500 bp in length, each of the one or more known synthetic DNA sequences including at least two primer binding sites and at least one discrete informational unit. Each discrete information unit may be between 2 bp and 100 bp in size. Authentication of the one or more discrete informational units may be accomplished via the use of one or more sequence specific DNA detection technologies. Sequence specific DNA detection technologies may include, without limitation, next generation sequencing, Sanger sequencing, nanopore sequencing, quantitative PCR (qPCR), reverse transcription PCR (RT-PCR), hybridization probes, nucleic acid probes such as Taqman probes, molecular beacons, peptide, nucleic acid (PNA)-based probes, micro-arrays and/or any other sequence specific detection technology. A microarray may use one or more of the foregoing sequence specific DNA detection technologies on the same array.

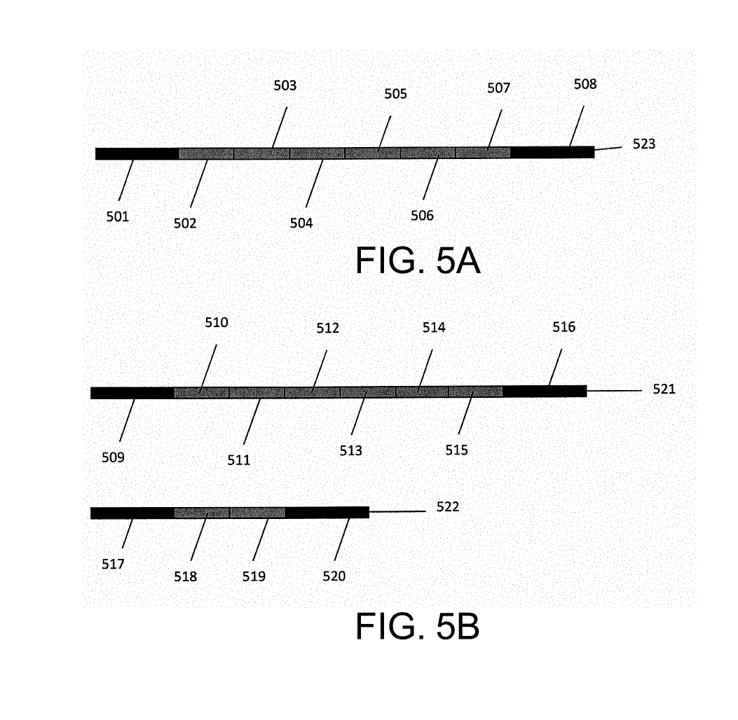

FIG. 5A shows an exemplary DNA tag. The DNA tag (523) comprises two terminal primer binding regions (501 and 508) and multiple discrete informational units (502-507). The total DNA tag length can be between 75 and 500 bp. In an exemplary embodiment, the DNA tag is between 150 bp and 300 bp in length. Each discrete information unit (502-507) is configured to impart a single piece of data. The data may include, without limitation, whether the cannabis plant was grown under a legally obtained license, the country of growth, the state of growth, processor identification, date of harvest, and/or the identification of a specific grower, process and/or distributor.

FIG. 5B shows an embodiment of a combinatorial DNA tag. The combinatorial DNA tag comprises two known DNA sequences (521 and 522) of different lengths. The larger DNA sequence (521) may vary in size from 100 bp to 500 bp and comprises at least two primer binding regions (509 and 516) and one or more discrete information units (510-515). The larger known DNA sequence (521) may be an amplicon. The amplicon may be produced via PCR. The larger DNA sequence's discrete information units (510-515) impart high-level (low specificity) information about a cannabis or cannabis derivate product such as, for example, whether the plant was grown under a legally obtained license, the country of growth, the state of growth and/or county of growth. These pieces of high-level information are common to several different geographically similar stakeholders in a cannabis supply chain. By way of example, two or more legal cannabis stakeholders with close geographical proximity can use the same known DNA sequence (521) since the data comprising their growth under a legally obtained license, the country of growth and the state of growth would be identical. Due to this fact, the larger known DNA sequence (521) may be an amplicon. Amplicons can be mass-produced via PCR amplification or other enzymatic based amplification events or reactions including, without limitation, the polymerase chain reaction (PCR), loop mediated isothermal amplification, rolling circle amplification, nucleic acid sequence base amplification, and ligase chain reaction or recombinase polymerase amplification.

The shorter known DNA sequence (522) exemplified in FIG. 5B may be of a length between 25 bp and 100 bp. The shorter known DNA sequence (522) may be an amplicon or it may be an oligonucleotide produced by oligonucleotide synthesis and comprised of at least one primer binding region (517 and 520) and one or more discrete information units (518 and 519). In an exemplary embodiment, the shorter known DNA sequence (522) is an oligonucleotide of approximately 75 bp in length, said oligonucleotide's discrete information units (518 and 519) imparting low-level (high specificity) data relating to a cannabis product or cannabis derivative product such as, for example, the identity of a specific grower, the identity of a specific processor, the identity of specific distributor, or date of tag application. These pieces of low-level data are not shared between cannabis stakeholders. The primer binding regions of the two known DNA sequences (521 and 522) may be identical, or may be different, such that specific interrogation of only one of the sequences within the DNA tag is possible.

Through the use of a DNA tag comprising a combination of a larger amplicon of a known DNA sequence imparting high-level data (521) produced by PCR, and a shorter oligonucleotide of a known sequence imparting low-level data (522) produced by oligonucleotide synthesis, it is possible to manufacture highly-specific and customizable DNA tags at large scale at low cost.

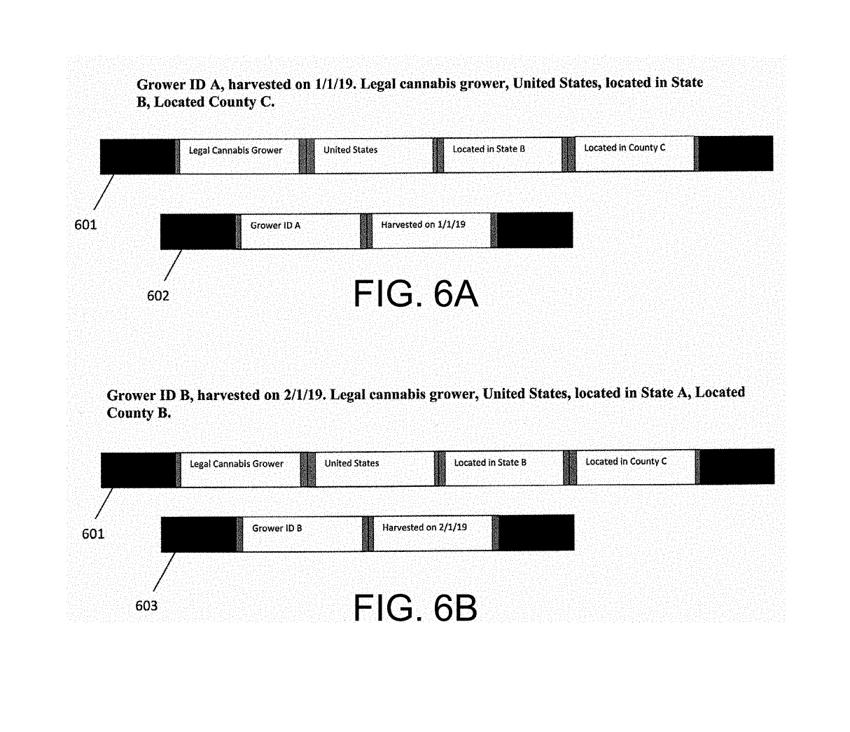

FIG. 6A and FIG. 6B show two different exemplary DNA tags imparting different information upon authentication for two different cannabis products. FIG. 6A shows a combinatorial DNA tag imparting the following information: (a) cannabis grower ID of “A”; (b) the cannabis product was harvested in Jan. 1, 2019; (c) the cannabis product is from a legal cannabis grower; (d) the cannabis product was grown in the United States; (e) the cannabis product was grown in state “B”; and (f) the cannabis product was grown in county “C”. The high-level data, comprised of the foregoing data points (c) through (f) are imparted by discrete information units on the larger amplicon (601). The low-level data, comprised of the aforementioned data points (a) and (b), are imparted by discrete information units on the shorter oligonucleotide (602).

FIG. 6B shows an exemplary DNA tag imparting the following information: (m) cannabis grower ID of “B”; (n) the cannabis product was harvested on Feb. 1, 2019; (o) the cannabis product is from a legal cannabis grower; (p) the cannabis product was grown in the United States; (q) the cannabis product was grown in state “B”; and (r) the cannabis product was grown in county “C”. The high-level data, comprised of the foregoing data points (o) through (r), are imparted by the identical amplicon (601) shown in FIG. 6A, since the high-level information is identical. The low level-data, comprised of data points (m) and (n) are imparted by discrete information units on a different oligonucleotide (603) than the oligonucleotide (602) used in FIG. 6A. Thus, via this scheme of rational DNA tag design, the high-level data imparting amplicon (601) can be mass produced via PCR amplification or other enzymatic based amplification events at large scale and be used for more than one cannabis product and/or cannabis derivative product stakeholder. The low-level data necessary for each specific cannabis product and/or cannabis derivative product stakeholder can be added to the high-level information conveying amplicon via the manufacture and addition of a specific oligonucleotide imparting the desired low-level information.

According to one embodiment, a DNA tag may comprise an amplicon with one or more discrete informational units and more than one oligonucleotide, each oligonucleotide comprising one or more discrete informational units. Oligonucleotides with one or more discrete informational units may be added to a cannabis product and/or cannabis derivative product at different points in the supply chain. Under this DNA tag scheme, additional low-level information is imparted to the cannabis products and/or cannabis derivative product via the addition of each oligonucleotide.

### Marking Apparatus [A1]

A marking apparatus may be used to apply the DNA tag to cannabis plant material. Application of the DNA tag to the cannabis plant material may take place at any point in the supply chain, including but not limited to the plant growth stage, cultivation state or drying stage. Application of a DNA tag may also occur after chemical extraction or concentration of cannabis material. Cannabis derivative products may be directly tagged. The marking apparatus may be comprised of a misting system, electrostatic spray system, atomized spray system, sprinkler system, fogging system, cooling system, aqueous solution dispensing apparatus, handheld spray device, powder dusting system, or any combination thereof configured to deliver DNA tags.

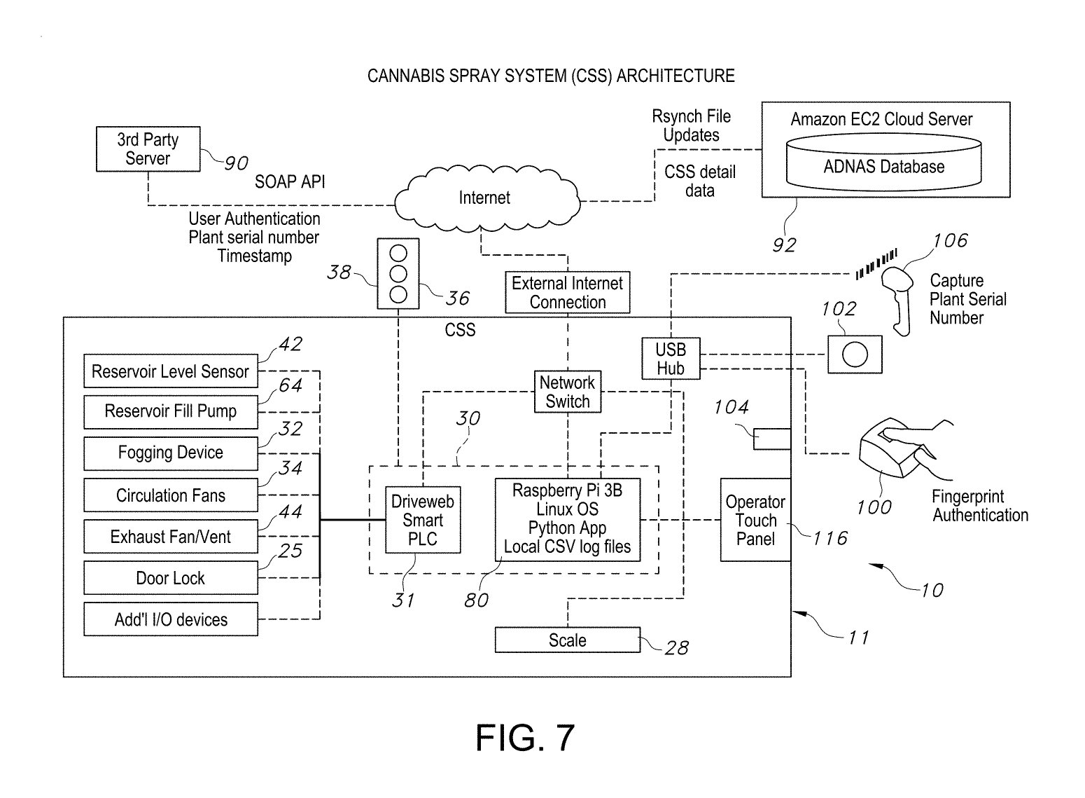

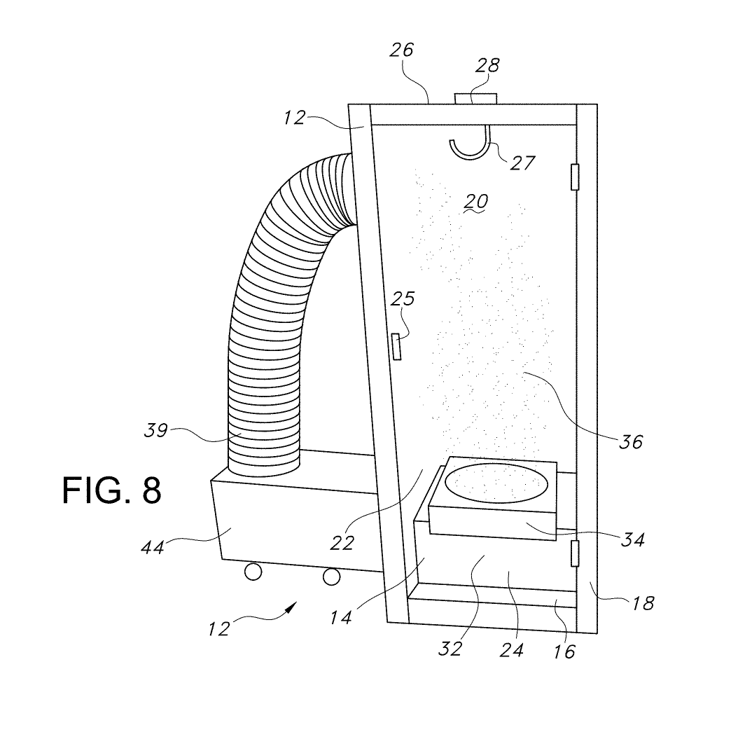

In an embodiment, the marking apparatus is a fogging system. In FIG. 7, [A2] a nucleic acid tag fogging (or “spraying”) system (10) is shown. The fogging system (10) may be designed to be used by growers of cannabis products for small and large operations and either indoor or outdoor growers. The fogging system (10) may be utilized indoors and operated in normal room temperatures. The fogging system (10) may be used to spray and/or atomized an aqueous nucleic acid tag solution (“taggant solution”) onto plant material (8), such as cannabis, with the nucleic acid tag solution containing known nucleic acid sequences with one or more discrete informational units. The nucleic acid tag may be a DNA tag comprised of an amplicon, or a DNA combinatorial DNA tag comprised of an amplicon and an oligonucleotide. While cannabis is noted herein as the plant material, it is within the contemplation of the present disclosure that the other types of plants or other non-plant material could be sprayed with the nucleic acid tag solution by the spraying system (10). The fogging system (10) is designed to consistently, safely and completely tag plant material with a nucleic acid tag through the use of a cold fog. In one exemplary embodiment, the system (10) may be able to fog a plant (8) through an entire cycle in less than approximately one minute and the system (10) is ready to remove the plant material.

The fogging system (10) may include a fogging apparatus (11) having fogging chamber (12) that can accommodate a cannabis plant with dimensions of about 48″ in diameter and 72″ tall. These dimensions are exemplary and not intended to be limiting as the chamber (12) could be of any size to accommodate the material to be tagged. The fogging chamber may be comprised of a sealed room, shipping container or any other defined space capable of being immersed in a fog. The fogging chamber (12) may be supported on a bottom base frame assembly (14). The base may include a space (16) for containing all the requisite equipment, infrastructure, system controller (30), control panel interface assembly (32) and wiring junction (34).

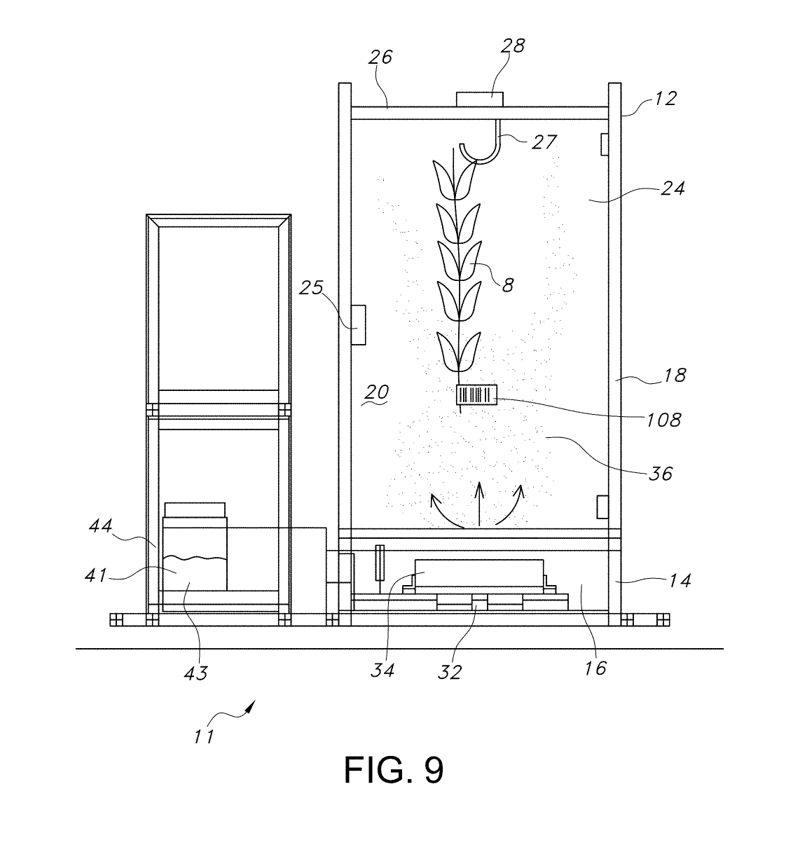

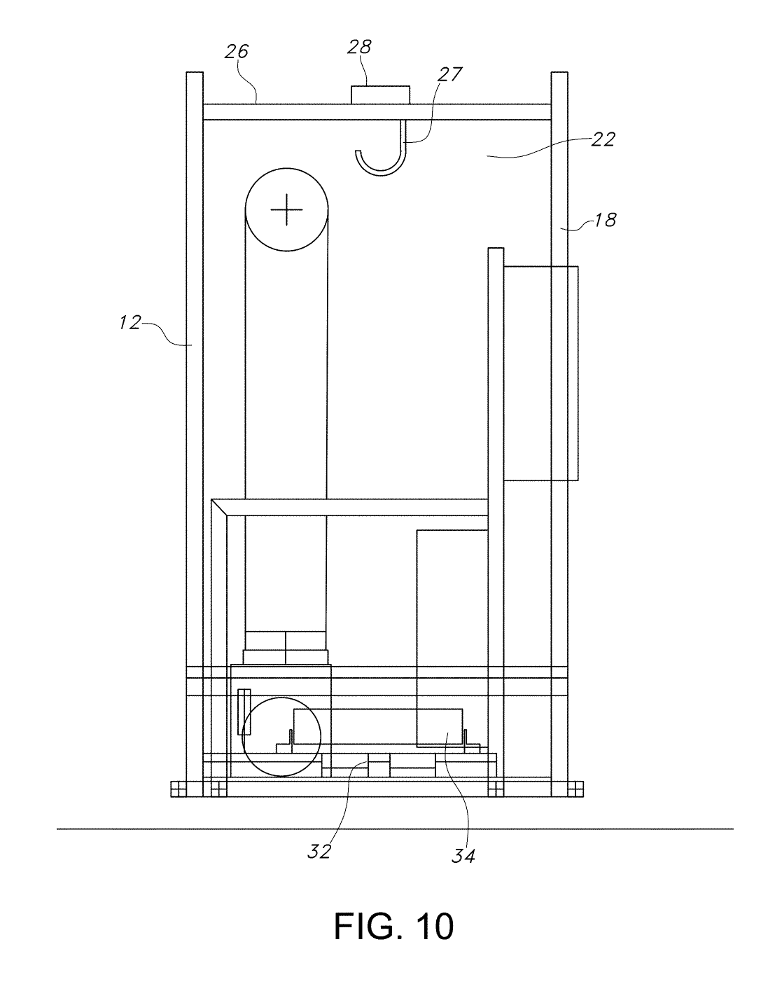

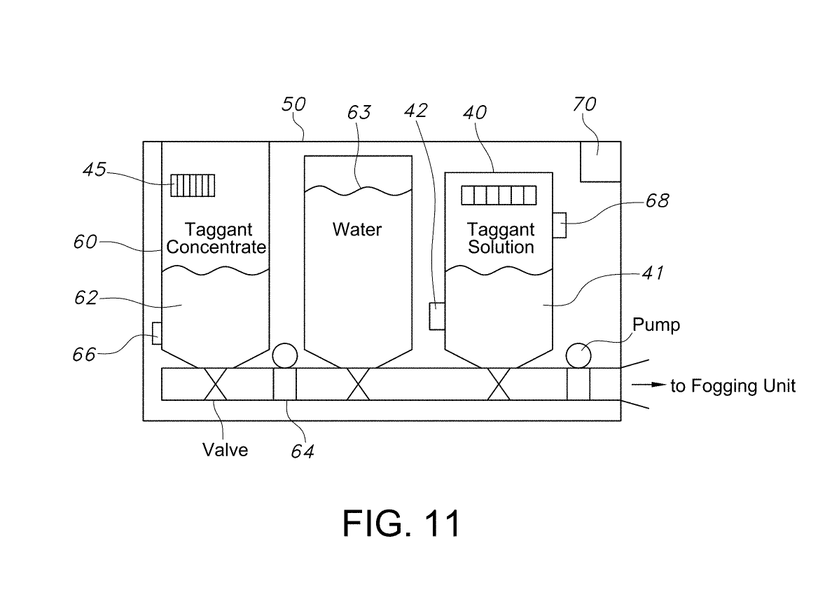

With reference to FIGS. 9-11, the fogging chamber (12) may include a plurality of side panels (18) joined at their edges forming a chamber interior (20). In an exemplary embodiment, the side panels (18) may be formed of transparent panels formed of clear LEXAN® and be approximately 48″ wide by 72″ high. One of the side panels (22) may include a door (24) to provide access into the chamber interior (20). The door (24) may be selectively secured by an electronic door lock mechanism (25) that is operated by the system controller (30). The system controller (30) may include a processing device (31) in the form of a programmable logic controller (PLC), such as Drive.web Smarty® PLC from Bardac Corporation of Stevensville, Md.

The chamber may include a top panel (26). A weighting scale (28) may be secured to the top panel (26) and include an attachment assembly (27), such as a hook (23), for hanging of the plant material thereto. The scale (28) determines the weight of the plant which is used in the tagging process and the system controller (30) records the weight. One or more fogging units (32) may be disposed in the base below a mist/fog dispersal device, such as a fan (34). The fogging units (32) may generate an atomized mist (36) using ultrasonic transducer(s) which vibrates rapidly at high speeds within the nucleic acid tag solution to form individual droplets, thereby producing a cool atomized mist carrying the nucleic acid tags. No heat is used to generate the mist, as heat can degrade the nucleic acid tag and/or harm the plant material. The generated atomized mist (36) is dispersed and circulated throughout the chamber interior by the fan (34) to completely coat the plant material (8) with the nucleic acid tag. The fan (34) may be operably connected to the processing device (31) and have variable speed adjustment. The spray chamber (12) may further include a display panel (36) including LED lighting (38), e.g., individual Red, Green, Yellow lights for highlighting cycle of operation. The fogging chamber (12) may further include a tank (40) that holds a nucleic acid tag solution (41) and is fluidly connected to the fogging units (32). A tank low level sensor (42) is provided to maintain the fluid level in the tank (40) to achieve optimal fogging conditions.

After the plant material (8) has been fogged for the requisite amount of time, the mist containing the nucleic acid tag (36) is removed from the spraying chamber (12) by an exhaust and filtration system (44). The exhaust and filtration system (44) removes the mist (36) and filters the nucleic acid tag out of the air stream. The exhaust system may include one or more fans and ductwork (39) which removes the atomized mist from the spraying chamber. The mist (36) may then be treated by a filter which separates the atomized nucleic acid tag solution from the air. The captured tag solution may be held in a tank (43) which is locked and only removable by authorized personnel. In this way, the nucleic acid taggant solution cannot be used to mark other items in an unauthorized manner, thus maintaining the integrity of the nucleic acid taggant solution. The exhaust and filtration system (44) may be connected to a dehumidifier such that the mist evacuated from the spray chamber (12) is condensed and collected in a collection tank in fluid communication with the dehumidifier. The collection tank may also be in fluid communication with the taggant solution tank (40) such that the condensed evacuated mist can be reused for additional tagging instances. The dehumidifier output may be operatively connected to the spray chamber (12) such that the dehumidified evacuated air from the spray chamber (12) is reintroduced into the spray chamber during the exhaust cycle. In this manner, any remaining nucleic acid taggant in the dehumidified exhausted air is not released to the atmosphere and is reintroduced into the spray chamber.

In one exemplary embodiment, the filtered nucleic acid tags may be treated to render the taggant inoperable to be used for further identification purposes. For example, the taggant solution could be subjected to heat or ultraviolet light which would degrade and/or denature the nucleic acid tags. Subjecting the taggant solution to a chemical such as an oxidizing agent, e.g., bleach, may also be used. In this way, the nucleic acid taggant solution could not be used in an unauthorized matter and attached to another item outside of the verification system.

A nucleic acid taggant concentrate reservoir (60) is fluidly connected to a taggant concentrate pump (64). In one exemplary embodiment, the pump may operate at a consistent rate (i.e. 1 ml/sec). The pump may be under PLC control (31) and may operate at set durations to deliver a required volume of taggant concentrate per batch (i.e. if 30 ml of taggant concentrate are needed the pump will be on for 30 seconds). A taggant concentrate low level sensor (66) is provided in the nucleic acid taggant concentrate reservoir (60) to indicate when additional nucleic acid taggant concentrate (62) needs to be added. A nucleic taggant solution (41), used to fog the plant material, is prepared by the fogging system (10) and is comprised of nucleic acid taggant concentrate and water. The taggant solution tank (40) may store approximately 1-6 liters of taggant solution. The tank (40) may locked so that the stored solution cannot be impermissibly accessed. The water may be supplied from a local source and controlled by an automated water valve. This water valve may be controlled by the PLC controller and allows plant water to flow in order to complete a batch of taggant solution. The fogging system (10) may include a reverse osmosis system disposed between the plant water source and the fogging system.

The preparation of a batch of nucleic acid taggant solution (41) may be initiated by the taggant solution tank low level sensor (42) sending a signal once this level is reached. A predetermined amount of nucleic acid taggant concentrate is then pumped into the taggant solution tank (40). The water valve is opened by the PLC (31) to allow water to flow into the taggant solution tank (40). The water valve is closed when a required predetermined amount of water has been added to the taggant solution tank. A taggant solution tank high level sensor (68) sends a signal when this high level is reached in the taggant solution tank (40) it signifies that enough plant water has been added and that the batch process is complete.

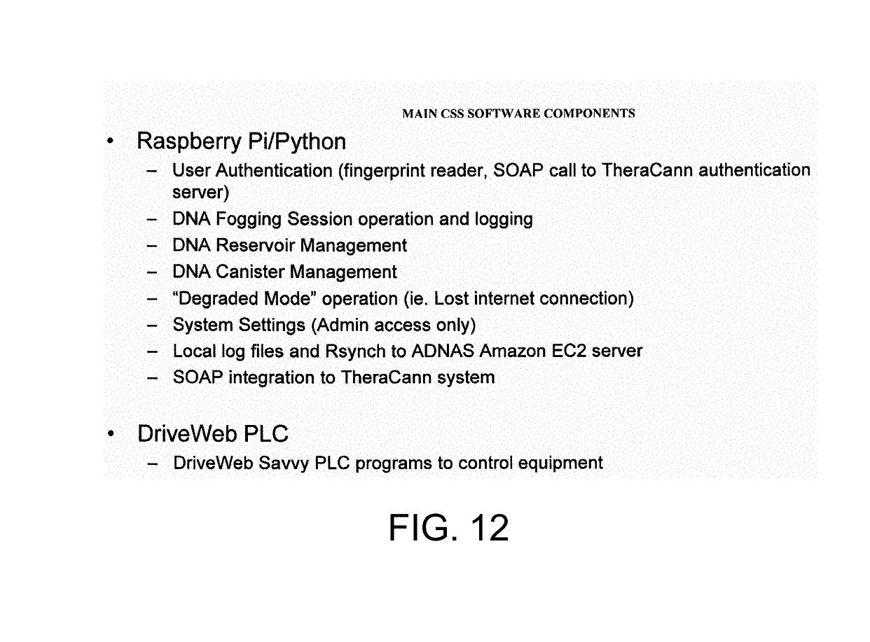

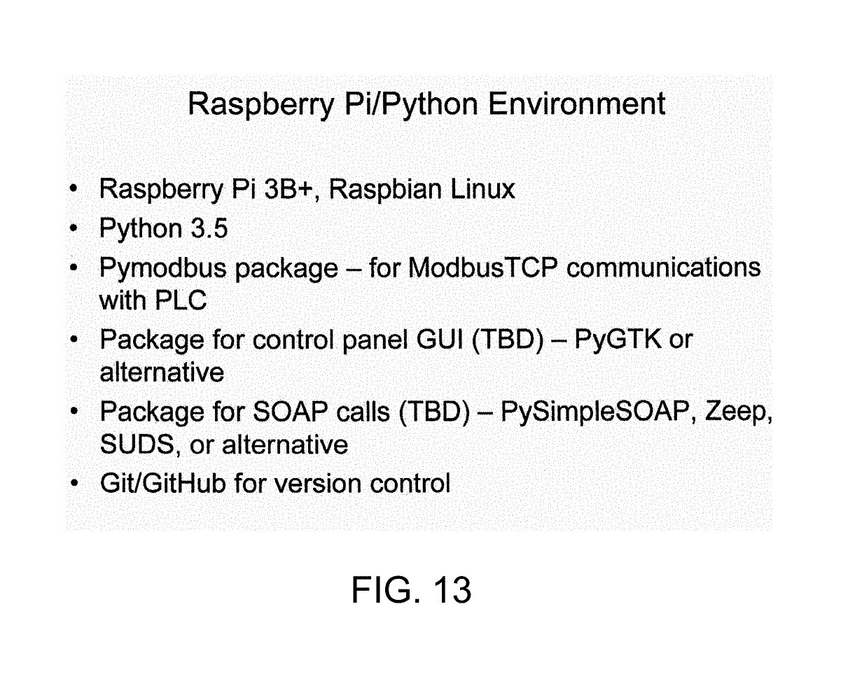

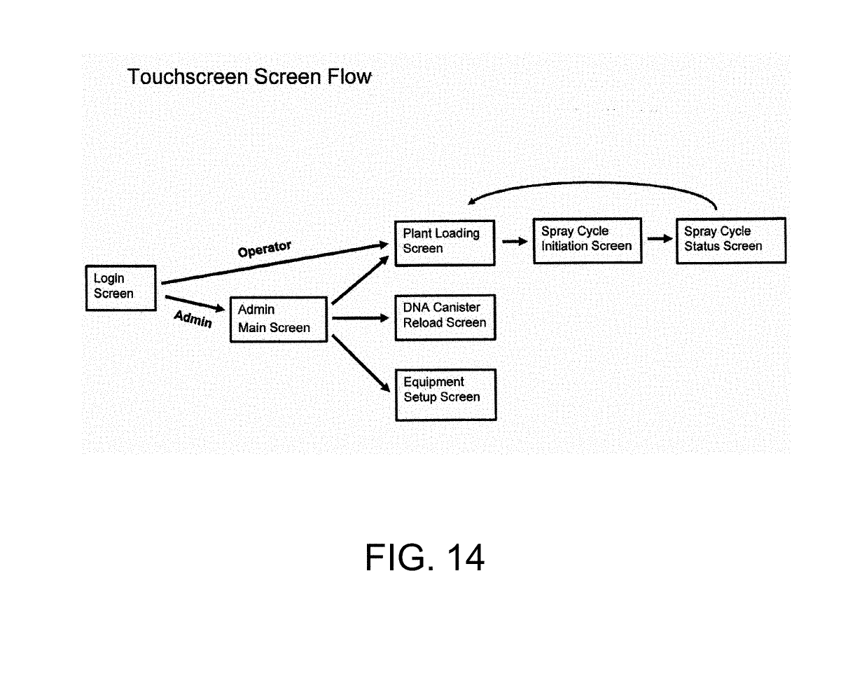

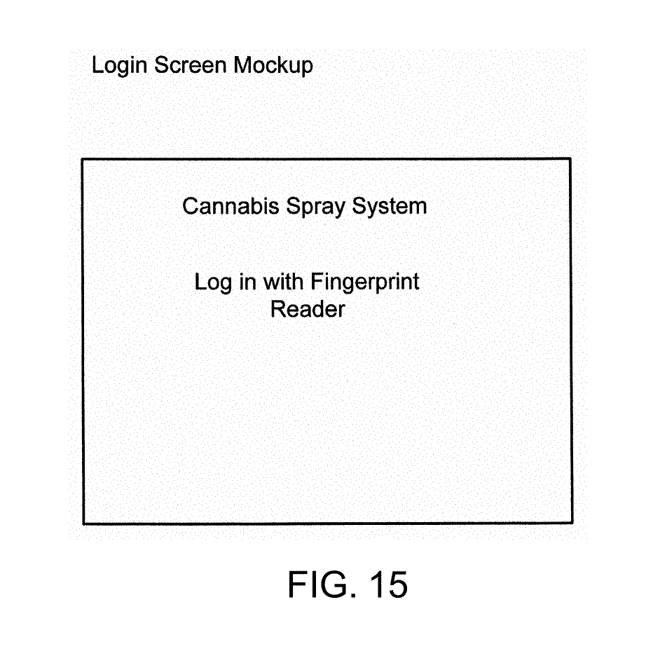

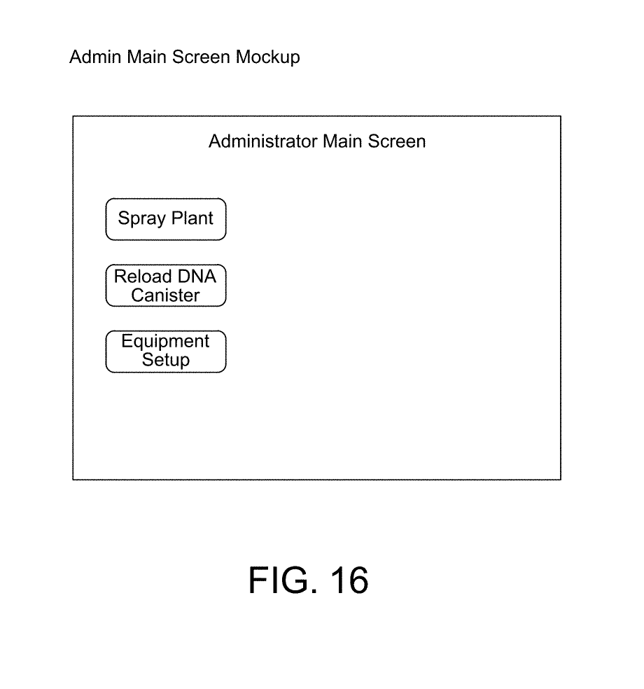

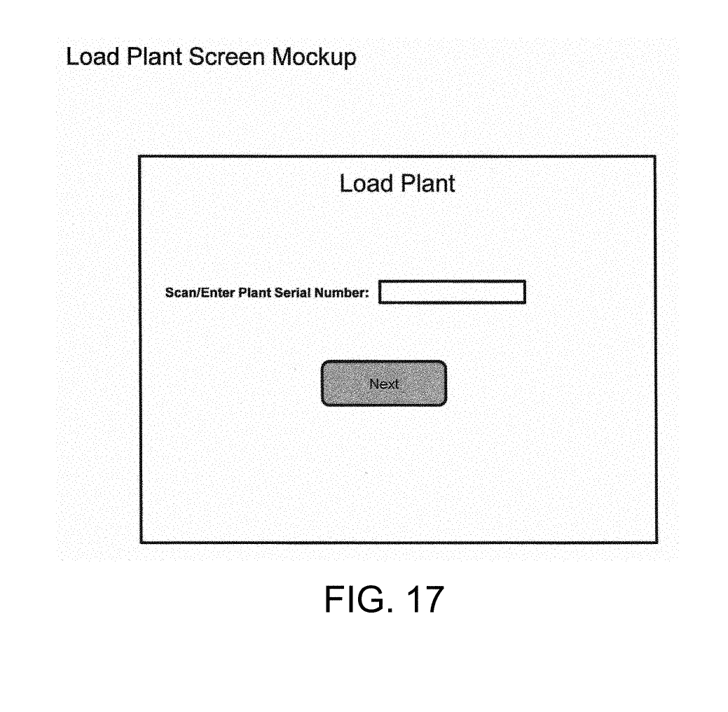

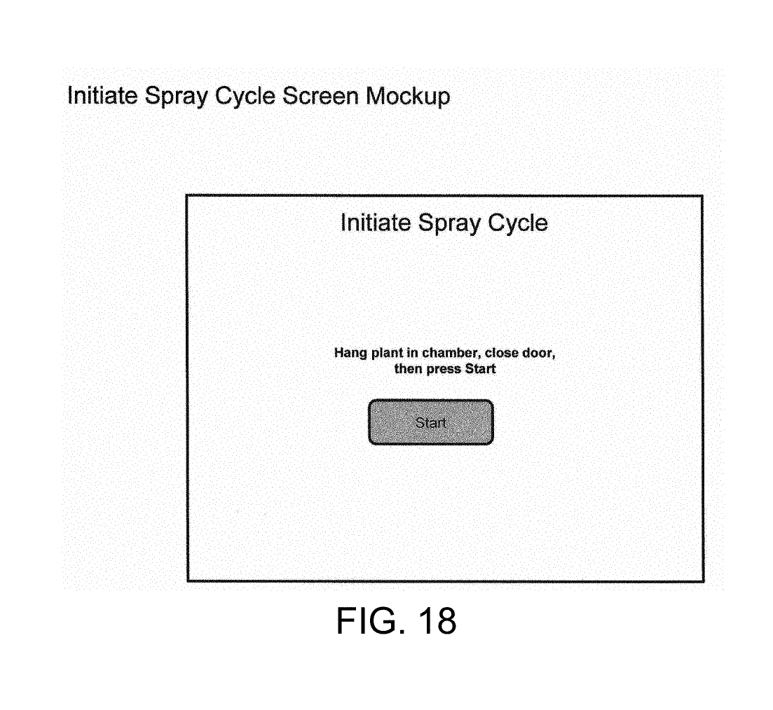

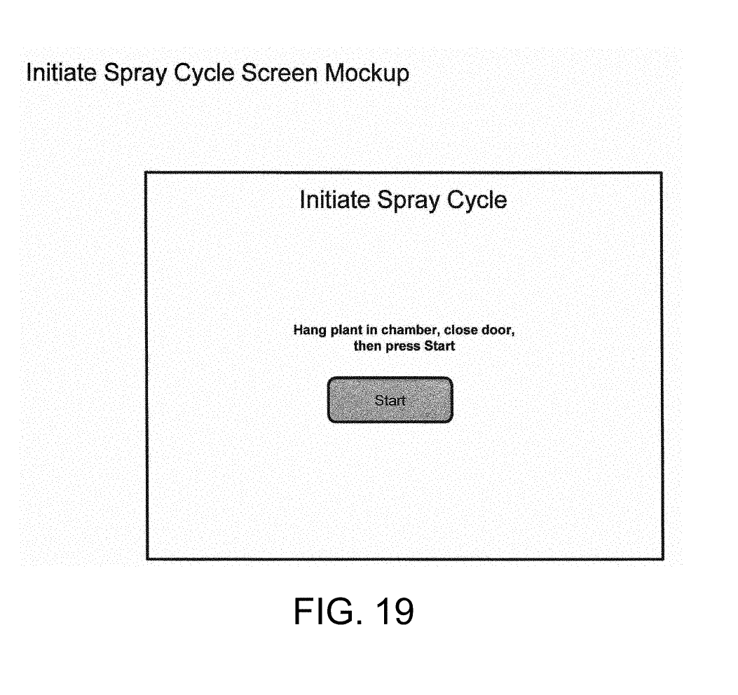

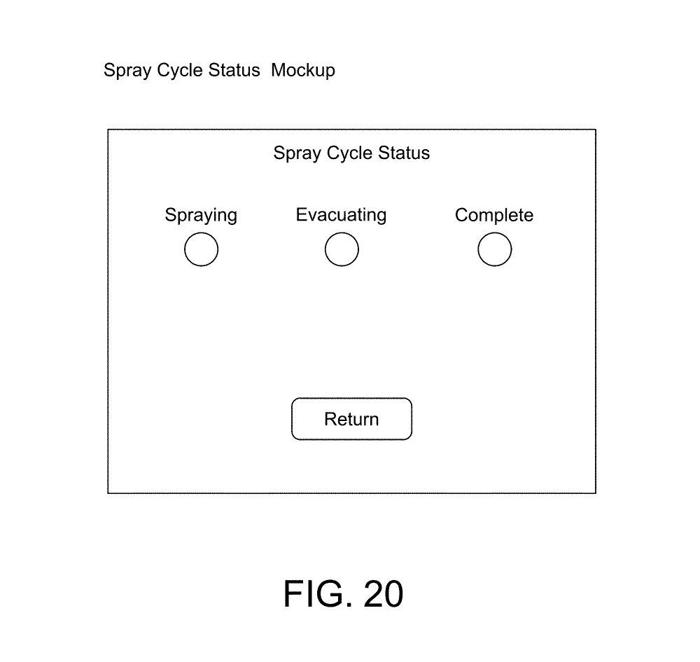

With further reference to FIGS. 12-13 and as noted above, the fogging (“spraying”) system (10) includes the system controller (30) disposed on the base frame (14). The system controller (30) controls all functions of the fogging system (10) and is programmable to work with all physical sensors, switches, lights, motors, pumps or other actuators for use with the fogging system (10). The system controller may also be configured to work with a reverse osmosis system. The system controller (30) includes the PLC (31) and an operator and software interface controller (80). The software interface controller (80) may be a computing device, such as a Raspberry PI computer that controls the visual interface for the operator as well as support all communications to the outside world, whether it be to a third-party supply chain party verification system/computer network (90) via APIs or to the system supplier's computer network (92). In addition, the software interface controller may control all elements of the spraying system (10) in the event of loss of Internet connectivity, thus allowing operation of the spraying system (10) by an operator.

The fogging system (10) may include a fingerprint accessory reader (100) for authentication of a user. Two-factor authentication may be a system option that can be selected. If invoked, authorization may be accomplished with the entering of a unique security code (or other digital asset unique to the individual) in addition to presenting finger for fingerprint identification. The spraying system (10) may further include a scanner/barcode scanner (106) such as a USB barcode scanner for Raspberry Pi. The scanner (106) may be used to scan the plant material barcode ID (108). This permits an operator to introduce a plant's information into the spraying system (10). The scanner may have a USB to serial interface to interface with the Raspberry Pi computer device (80). The scanner (106) may also be used to scan barcodes associated with the taggant concentrate.

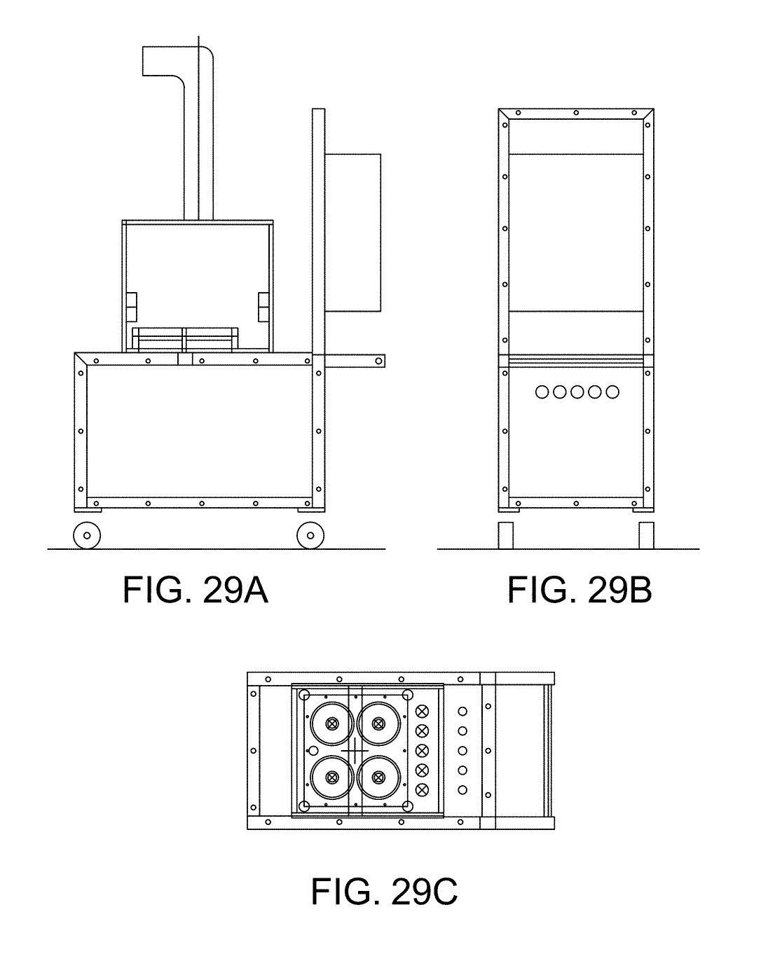

The fogging system may also be configured to tag plant material not disposed within a fixed enclosure attached to the unit. One or more fogging units may be disposed within a defined space (e.g. green house, drying room, shipping container of other room) such that the fog generated by the fogging units disperses throughout the room, thus tagging the contents of the room with the nucleic acid taggants contained within the fog. The defined space containing one or more fogging units may further include an exhaust and filtration system of identical functionality as described above. An exemplary embodiment of a fogging unit configured to tag plant matter not disposed within a fixed enclosure is shown in FIG. 29.

In one embodiment, the marking apparatus is digitally connected to the digital data repository and automatically updates the digital data repository with information relating to the marking apparatus' DNA tagging operations. This information may include DNA tag information (sequence, length, combinatorial mark composition), location, time, date, mass/weight tagged and other similar information. This information will be automatically digitally communicated to the digital data repository. The digital data repository will create a database of the information for subsequent query by the authentication apparatus and/or a user. For purposes of definition, a database may also be considered part of a blockchain implementation where distributed ledgers represent the entire database. The marking apparatus may be a node or nodes in a blockchain.

The marking apparatus may also be operatively connected to an oligonucleotide synthesis apparatus such that customized oligonucleotide sequences may be created on demand. Exemplary oligonucleotide synthesis apparatuses include the BioXp 3200 system from SGI-DNA (California, USA) and the MerMade system from BioAutomation (Texas, USA). In this embodiment, the oligonucleotide synthesis apparatus and marking apparatus may both be digitally connected to digital data repository. The oligonucleotide synthesis apparatus may also be operatively connected to a DNA amplification apparatus such as a thermocycler or isothermal DNA amplification system to generate a quantity of DNA tags. The oligonucleotide sequences created on demand may be mixed with pre-manufactured amplicons to create DNA tags comprised amplicons and oligonucleotides. The thermocycler may be a continuous flow thermocycler as disclosed in U.S. Pat. No. 8,163,489, Murry, et al.; U.S. Pat. 8,293,471, Gregg et al.; and/or U.S. Pat. No. 8,986,982, Gregg et al.

The marking apparatus may deliver a nucleic acid tag at various concentrations to a cannabis product, cannabis derivative product or other plant material. Suitable exemplary ranges of DNA tags utilized by a marking apparatus include for instance:

A range from about 0.1 nanogram (10⁻¹⁰ g) to about 10 microgram (10×10⁻⁶g) of DNA tag per kilogram (10³ g) of cannabis product, cannabis derivative product or other plant material.

A range from about 0.1 nanogram (10⁻¹⁰ g) to about 1 microgram (10⁻⁶g) of DNA tag added per kilogram (10³ g) of cannabis product, cannabis derivative product or other plant material.

A range from about 0.1 nanogram (10⁻¹⁰ g) to about 100 nanograms (100×10⁻⁹ g) of DNA tag added per kilogram (10³ g) of cannabis product, cannabis derivative product or other plant material.

A range from about 0.1 nanogram (10⁻¹⁰ g) to about 10 nanograms (10×10⁻⁹ g) of DNA tag added per kilogram (10³ g) of cannabis product, cannabis derivative product or other plant material.

A range from about 1 picograms (1×10⁻¹² g) to about 100 microgram (100×10⁻⁶ g) of DNA tag added per kilogram (10³ g) of cannabis product, cannabis derivative product or other plant material.

A range from about 1 femtogram (10⁻¹⁵ g) to about 1 microgram (10⁻⁶ g) of DNA tag added per kilogram (10³ g) of cannabis product, cannabis derivative product or other plant material.

A range from about 10 femtograms (10×10⁻¹⁵ g) to about 100 nanograms (100×10⁻⁹ g) of DNA tag added per kilogram (10³ g) of cannabis product, cannabis derivative product or other plant material.

A range from about 100 femtograms (100×10⁻¹⁵ g) to about 10 nanograms (10×10⁻⁹ g) of DNA tag added per kilogram (10³ g) of cannabis product, cannabis derivative product or other plant material.

A range from about 1 picograms (1×10⁻¹² g) to about 1 nanogram (1×10⁻⁹ g) of DNA tag added per kilogram (10³ g) of cannabis product, cannabis derivative product or other plant material.

Applying nucleic acid tags can be accomplished by any means by which tags are placed into contact with a plant and remain on the plant, such as by the aforementioned marking apparatuses. In some embodiments, nucleic acid tags can be applied by using marking apparatuses of a smaller scale, such as, for example, with eye droppers. Small scale application methods are typically used for marking derivative products.

### Authentication Apparatus

An authentication apparatus is used to interrogate DNA tagged cannabis products and/or cannabis derivative products, or other plant material. The authentication apparatus may be capable of extracting the data of one or more discrete informational units contained within a DNA tag. The authentication apparatus may be any apparatus capable of sequence specific DNA detection, and may include, without limitation, capillary electrophoresis apparatus, qPCR apparatus, PCR apparatus, microarray and microarray scanning apparatus, multiplex analysis apparatus, multi-mode reader, next generation sequencing apparatus, Sanger sequencing apparatus, microarray, nanopore sequencing apparatus, hybridization/nucleic acid probe detection apparatus, or any combination thereof. A microarray may use one or more of the foregoing sequence specific DNA detection technologies/apparatus on the on the same array.

In an embodiment, a specific qPCR assay is designed to extract data from a predefined discrete informational unit. In reference to FIG. 6A, specific qPCR assays may be designed to extract data from each of the informational units. For example, one qPCR assay may inform the user whether the cannabis product and/or cannabis derivative product was grown under a legal license. A separate qPCR assay may inform the user that the cannabis product and/or cannabis derivative product was grown in the United States. Yet another qPCR assay may inform the user that the cannabis product and/or cannabis derivative product was grown in state “B” or in county “C”. Still another set of qPCR assay's may be designed to interrogate the low-level data such as grower ID and harvest date. Each qPCR assay may be conducted separately on concurrently on a multi-channel qPCR device. A multi-mode reader, capable of detecting various wavelengths, and optionally, other types of non-florescent taggants or indicia, may be used to interpret the qPCR assays. An exemplary multi-mode reader is disclosed in U.S. Patent Application Publication US 2018/0173810 A1, Murrah, et al., published Jun. 21, 2018.

The discrete informational units of a DNA tag may also be interrogated via microarray wherein the microarray's unique hybridization/florescence profile would impart data from multiple discrete informational units simultaneously resulting in a unique microarray signature for each DNA tag. A microarray may be interrogated by a multi-mode reader. The discrete informational units may also be interrogated via next generation and/or Sanger sequencing. In one embodiment, the high-level information contained in the amplicon (601) may be interrogated via one or more qPCR assays, while the low level information contained in the oligonucleotide (602) may be interrogated via next generation and/or Sanger sequencing.

The authentication apparatus may be digitally connected to the digital data repository and configured to compare the DNA tag information and/or data from the tag's discrete informational units ascertained from a tagged cannabis product and/or cannabis derivative product with the database of DNA tags and associated information residing on the digital data repository. In this fashion, the DNA tag information detected by the authentication device will be automatically matched to DNA tag information residing on the digital data repository and the information associated with the identified DNA tag stored on the database will provide the origin of the sampled cannabis products and/or cannabis derivative product. In one embodiment, a microarray with a microarray scanning apparatus digitally connected to the digital data repository is utilized to ascertain the DNA tag information. The microarray scanning apparatus may be a multi-mode reader.

The microarray may include an amplification step that amplifies the DNA tag prior to introduction to the microarray. The microarray amplification step may include any known means of DNA amplification including but not limited to PCR, isothermal amplification, loop mediated isothermal amplification, rolling circle amplification, ligase chain reaction, or recombinase polymerase amplification (RPA). The microarray may be configured to be readable under visible or UV light, without or without a microarray scanning apparatus. The microarray scanning apparatus may include a cell phone or tablet computer with a digital camera sensor, said cell phone or tablet running a software application configured to interpret a digital camera image of the microarray and digitally connect to the digital data repository. The cell phone or tablet computer may also have a visible or UV light source. The microarray scanning apparatus may be a multi-mode reader. In another embodiment, a qPCR device that is digitally connected to the digital data repository is utilized to ascertain the DNA tag information. The qPCR device may be a multi-mode reader.

The microarray may be created via a microarray printing apparatus also digitally connected to the digital data repository. In this embodiment, the microarray printing apparatus will automatically create custom microarrays, based upon the DNA tags dispensed by the marking apparatus, for the detection of one or more of the dispensed DNA tags. Exemplary microarray printing apparatuses include the NanoPrint 2 or SpotBot 3 by Arrayit Corp., or PersonalArrayer 16 by Core Life Sciences.

### Digital Data Repository

The digital data repository may be any known multi-user assessable digital storage means known in the art. In one embodiment, the digital data repository may be built upon cloud computing architecture, wherein the data is store on remote servers and accessed via the internet. In another embodiment, the digital data repository is based on a shared continually reconciled distributed database blockchain architecture. The blockchain may be public or private. Exemplary blockchain frameworks for the digital data repository include Hyperledger, Everledger or other public or private blockchain. In one embodiment, the marking apparatus, authentication apparatus, oligonucleotide synthesis apparatus and/or the users are nodes in the blockchain. In another embodiment, any combination of a marking apparatus, authentication apparatus, oligonucleotide synthesis apparatus and/or the users are nodes in the blockchain.

### Method for Tracking Cannabis Products and Cannabis Derivative Products

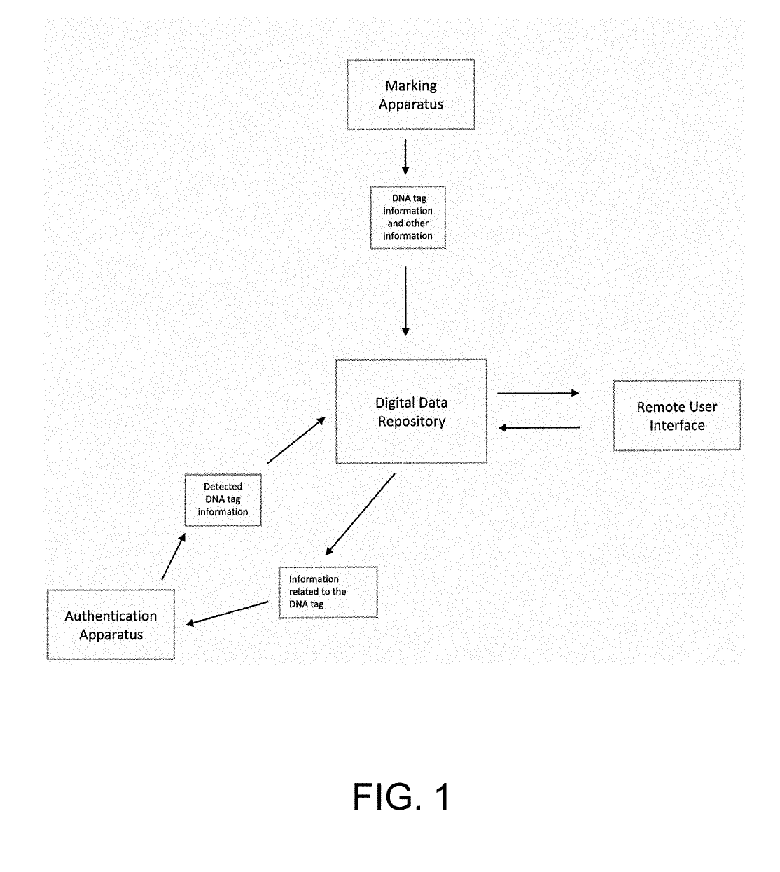

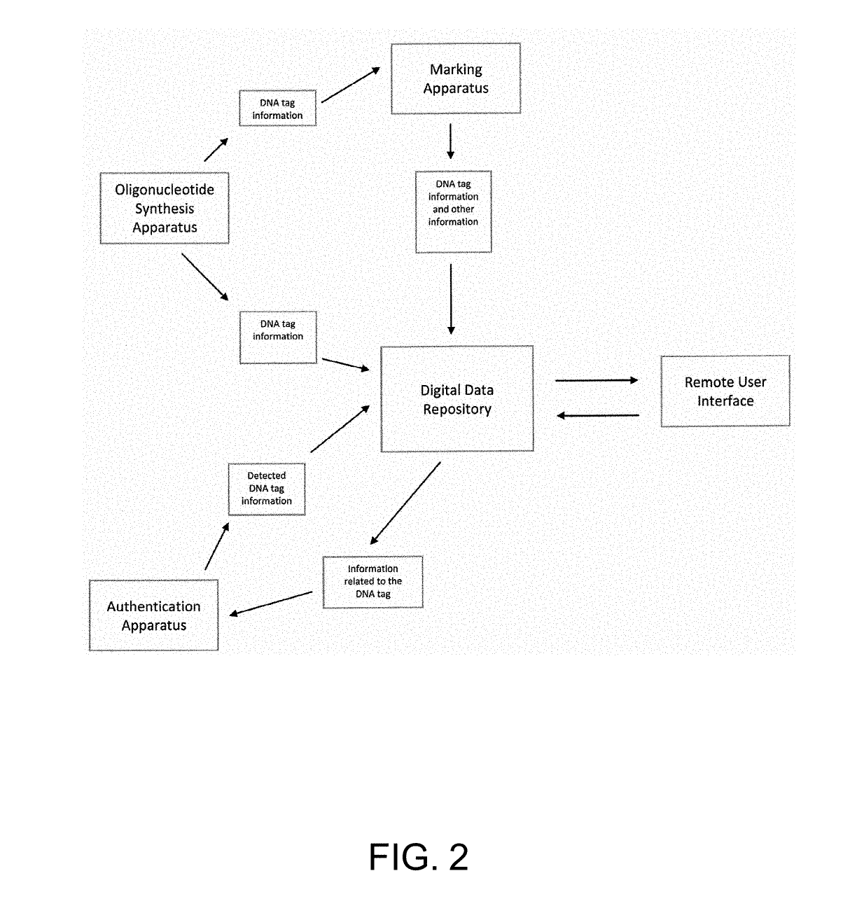

A method for the tracking the origin of cannabis products and cannabis derivative products is provided. As shown in FIGS. 1 and 2, the method may comprise: (a) applying a nucleic acid tag to a cannabis plant to create a nucleic acid tagged cannabis plant, said tag imparting at least one piece of information about the origin of the cannabis plant; (b) processing the nucleic acid tagged cannabis plant to create nucleic acid tagged cannabis products and/or nucleic acid tagged cannabis derivative products; and (c) interrogating a nucleic acid tagged cannabis product and/or cannabis derivative product to ascertain information from the nucleic acid tag; and optionally, (d) querying a digital data repository for information associated with the nucleic acid tag information; and (e) utilizing the nucleic acid tag information in conjunction with the information from the digital data repository to ascertain the source of the cannabis product and/or cannabis derivative product. The methods can also be used for tracking other types of plants.

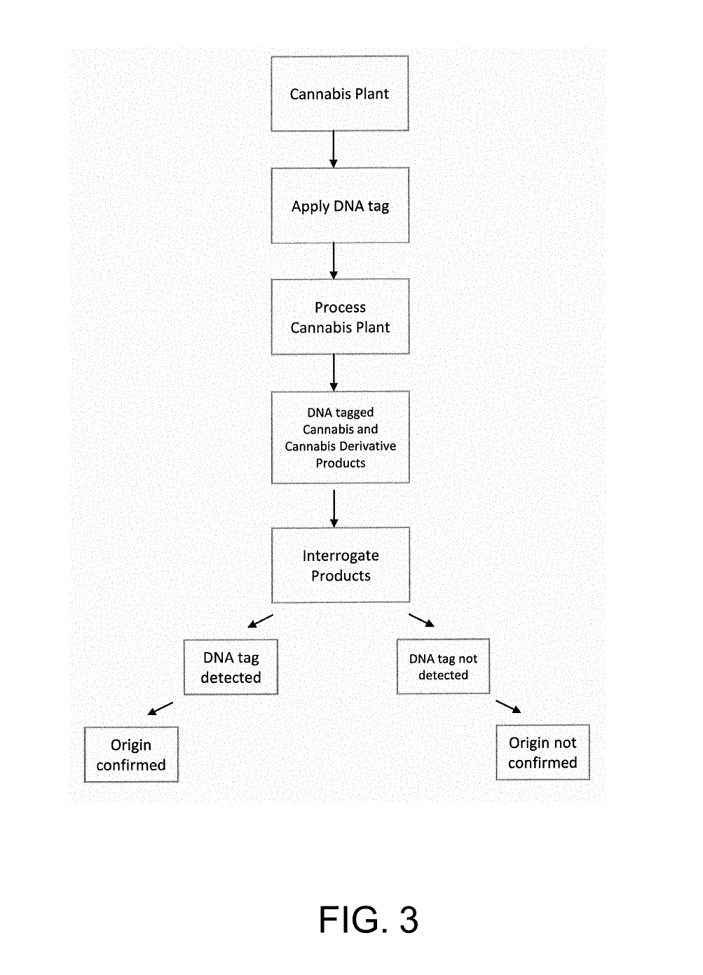

The nucleic acid tag may be a DNA tag (“taggant”). The nucleic acid tags can be applied by using the marking apparatuses described herein or by other means known in the art, for example, by small scale apparatuses, such as application by hand, or small application tools, such as, for example, an eye dropper; small scale application methods are typically used for marking derivative products. In one embodiment shown in FIG. 3, the DNA tag is applied to a live cannabis plant during the plant's growth stage. The plant's growth stage includes any time before harvest, for example, before maturation. The DNA tag can be applied to any part of the plant, including, for example, leaves, stems, buds and crowns. In other embodiment, the DNA tag is applied to a cultivated cannabis plant immediately after harvest or during the drying or other processing stage. The DNA tag may be applied to a live or cultivated cannabis plant via a misting system, electrostatic spray system, sprinkler system, fogging system, a cooling system, atomized spray system, pressurized spray system, handheld spray device, powder dusting system, or any combination thereof.

Once tagged with a DNA tag, the nucleic acid tagged cannabis plant is processed via known cannabis processing methods to create either cannabis products or cannabis derivative products tagged with the DNA tag. The DNA tag subsists through conventional cannabis processing without the need for reapplication. Cannabis processing methods include, for example, extraction methods and filtration methods, and any methods that are required to produce derivative products, e.g., products that can be derived from cannabis' essential oils, such as tinctures, transdermal patches, oral tablets and “gummies,” baked goods, vaporizing and dabbing oils. Examples of extraction methods include alcohol extraction, supercritical carbon dioxide extraction (SCCO₂), hydrocarbon extraction (e.g., butane extraction, propane extraction), and solvent free extraction (e.g., grinding and sieving).

Interrogation of the DNA tagged cannabis products and/or cannabis derivative products is undertaken via an authentication apparatus. The interrogation both confirms the presence of a DNA tag and ascertains information from the DNA tag that can be used, in conjunction with a database residing on digital data repository, to identify the origin of the cannabis product and cannabis derivative product. The information on the DNA tag may be stored within one or more discrete information units contained with the DNA tag.

### Block Diagram of a General Computer System

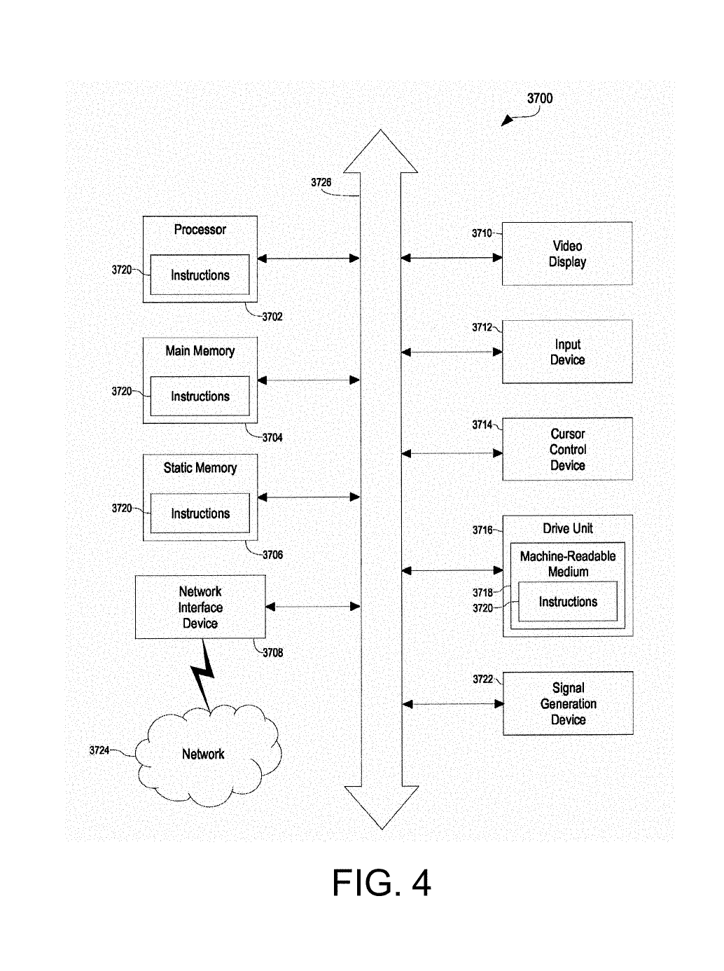

FIG. 4 is a block diagram of an illustrative embodiment of a general computer system 3700. The computer system 3700 can include a set of instructions that can be executed to cause the computer system 3700 to perform any one or more of the methods or computer based functions disclosed herein in FIGS. 1-3. The computer system 3700, or any portion thereof, may operate as a standalone device or may be connected, e.g., using a network or other connection, to other computer systems or peripheral devices. For example, the computer system 3700 may be the Digital Data Repository or the Remote User Interface, and may further be connected to other systems and devices, such as Marking Apparatus or Authentication Apparatus, via a network.

The computer system 3700 may also be implemented as or incorporated into various devices, such as a personal computer (PC), a tablet PC, a personal digital assistant (PDA), a mobile device (e.g., smartphone), a palmtop computer, a laptop computer, a desktop computer, a communications device, a control system, a web appliance, or any other machine capable of executing a set of instructions (sequentially or otherwise) that specify actions to be taken by that machine. Further, while a single computer system 3700 is illustrated, the term “system” shall also be taken to include any collection of systems or sub-systems that individually or jointly execute a set, or multiple sets, of instructions to perform one or more computer functions.

As illustrated in FIG. 4, the computer system 3700 may include a processor 3702, e.g., a central processing unit (CPU), a graphics-processing unit (GPU), or both. Moreover, the computer system 3700 may include a main memory 3704 and a static memory 3706 that can communicate with each other via a bus 3726. The memory may include information relating to DNA tag information. As shown, the computer system 3700 may further include a video display unit 3710, such as a liquid crystal display (LCD), an organic light emitting diode (OLED), a flat panel display, a solid state display, or a cathode ray tube (CRT). Additionally, the computer system 3700 may include an input device 3712, such as a keyboard, and a cursor control device 3714, such as a mouse. The computer system 3700 can also include a disk drive (or solid state) unit 3716, a signal generation device 3722, such as a speaker or remote control, and a network interface device 3708.

In a particular embodiment or aspect, as depicted in FIG. 4, the disk drive (or solid state) unit 3716 may include a computer-readable medium 3718 in which one or more sets of instructions 3720, e.g., software, can be embedded. Further, the instructions 3720 may embody one or more of the methods or logic as described herein. In a particular embodiment or aspect, the instructions 3720 may reside completely, or at least partially, within the main memory 3704, the static memory 3706, and/or within the processor 3702 during execution by the computer system 3700. The main memory 3704 and the processor 3702 also may include computer-readable media.

In an alternative embodiment or aspect, dedicated hardware implementations, such as application specific integrated circuits, programmable logic arrays and other hardware devices, can be constructed to implement one or more of the methods described herein. Applications that may include the apparatus and systems of various embodiments or aspects can broadly include a variety of electronic and computer systems. One or more embodiments or aspects described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments or aspects, the methods described herein may be implemented by software programs tangibly embodied in a processor-readable medium and may be executed by a processor. Further, in an exemplary, non-limited embodiment or aspect, implementations can include distributed processing, component/object distributed processing, and parallel processing. Alternatively, virtual computer system processing can be constructed to implement one or more of the methods or functionality as described herein.

It is also contemplated that a computer-readable medium includes instructions 3720 or receives and executes instructions 3720 responsive to a propagated signal, so that a device connected to a network 3724 can communicate voice, video or data over the network 3724. Further, the instructions 3720 may be transmitted or received over the network 3724 via the network interface device 3708.

While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a particular non-limiting, example embodiment or aspect, the computer-readable medium can include a solid-state memory, such as a memory card or other package, which houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals, such as a signal communicated over a transmission medium. A digital file attachment to an e-mail or other self-contained information archive or set of archives may be considered a distribution medium that is equivalent to a tangible storage medium. Accordingly, any one or more of a computer-readable medium or a distribution medium and other equivalents and successor media, in which data or instructions may be stored, are included herein.

In accordance with various embodiments or aspects, the methods described herein may be implemented as one or more software programs running on a computer processor. Dedicated hardware implementations including, but not limited to, application specific integrated circuits, programmable logic arrays, and other hardware devices can likewise be constructed to implement the methods described herein. Furthermore, alternative software implementations including, but not limited to, distributed processing or component/object distributed processing, parallel processing, or virtual machine processing can also be constructed to implement the methods described herein.

It should also be noted that software that implements the disclosed methods may optionally be stored on a tangible storage medium, such as: a magnetic medium, such as a disk or tape; a magneto-optical or optical medium, such as a disk; or a solid state medium, such as a memory card or other package that houses one or more read-only (non-volatile) memories, random access memories, or other re-writable (volatile) memories. The software may also utilize a signal containing computer instructions. A digital file attachment to e-mail or other self-contained information archive or set of archives is considered a distribution medium equivalent to a tangible storage medium. Accordingly, a tangible storage medium or distribution medium as listed herein, and other equivalents and successor media, in which the software implementations herein may be stored, are included herein.

In the event of a conflict between a definition recited in this specification and a definition provided in a patent or publication incorporated herein by reference, the definition provided herein is intended.

The disclosures of each of the references, patents and published patent applications disclosed herein are each hereby incorporated by reference herein in their entireties. However, the disclosure of a reference herein should not be construed as an acknowledgement that such reference is prior art to the present invention.

While the invention has been shown and described with reference to exemplary embodiments thereof, it will be understood by those of ordinary skill in the art that various changes in form and detail may be made therein without departing from the spirit and scope of the invention.

Examples have been set forth below for the purpose of illustration and to describe the best mode of the invention at the present time. The scope of the invention is not to be in any way limited by the examples set forth herein.

### EXAMPLES

### #1—Tagging of Cannabis Products and Cannabis Derivative Products

A sub 200 base pair DNA tag of a known sequence was placed into an aqueous solution and sprayed onto the leaves, stems and crowns of three cannabis plants just prior to plant maturation, and before plant cultivation and processing. Samples of the DNA tag (S1, S2 and S3) were taken at different time periods after application. S1 was taken on the date of DNA tag application. S2 was taken one week after DNA tag application, just prior to the plant curing process. Sample S3 was taken after the plant curing process. As seen below, all samples showed successful DNA tag recovery. All testing was performed via qPCR analysis on surface rinse samples.

The three DNA tagged cannabis plants were then used to produce cannabis derivative products via unaltered known methodologies. The produced cannabis derivative products were cannabis oil, cannabis budder, cannabis butane extract and Rick Simpson Oil (RSO).

Once produced via conventional processing, each of the foregoing cannabis derivative materials was extracted in triplicate with “E & D” buffer. The extracted solutions from each material was tested “in-field” through the use of a mobile qPCR device (MyGo PCR system, IT-IS Life Scienec, Ltd.). Samples of all materials also underwent laboratory analysis after concentration with ChargeSwitch magnetic beads (ThermoFisher Scientific).

All samples were vortexed vigorously for 2 minutes after incubation and the addition of dilution buffer and were spun to separate the resulting oil layer with the aqueous layer, where the DNA tag resided. The aqueous layer was then tested for the DNA tag applied to the cannabis plants during its growth stage. An appropriate number of PCR tubes were prepared. 18 μL of qPCR reaction mix was added into each tube, to which 2 μL of each extraction solution was added. The PCR tubes were then loaded into the MyGo qPCR instrument running real time DNA analysis with a 2-step amplification protocol for 40 cycles. The results, below, showed successful detection of the DNA tag in all cannabis derivative materials.

Results from laboratory qPCR analysis after sample concentration with ChargeSwitch magnetic beads likewise showed successful detection of the DNA tag, with at higher DNA concentrations than in-field analysis without concentration.

### #2—Nucleic Acid Tag Application Via Fogging

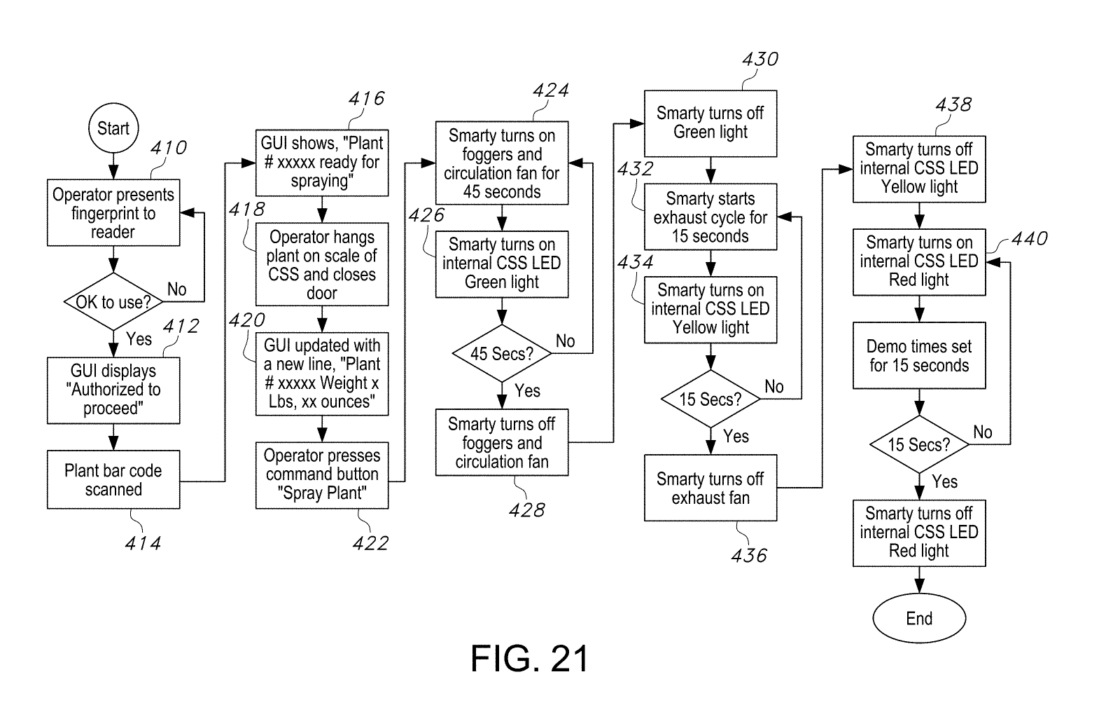

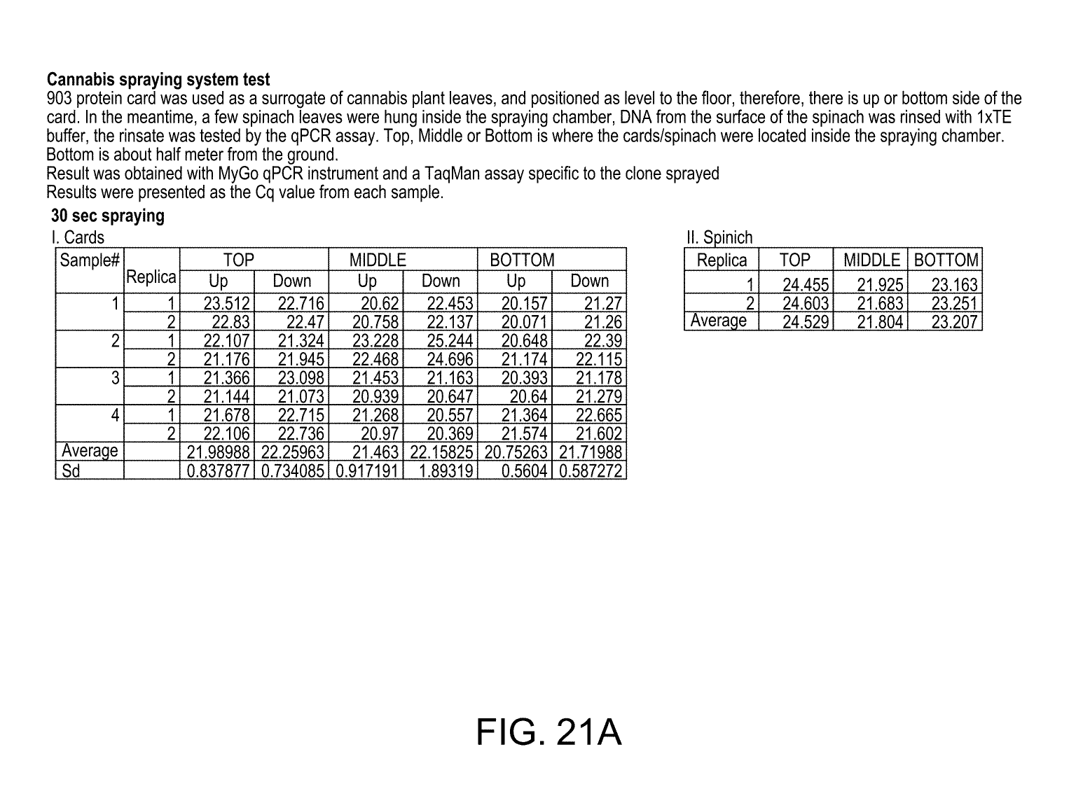

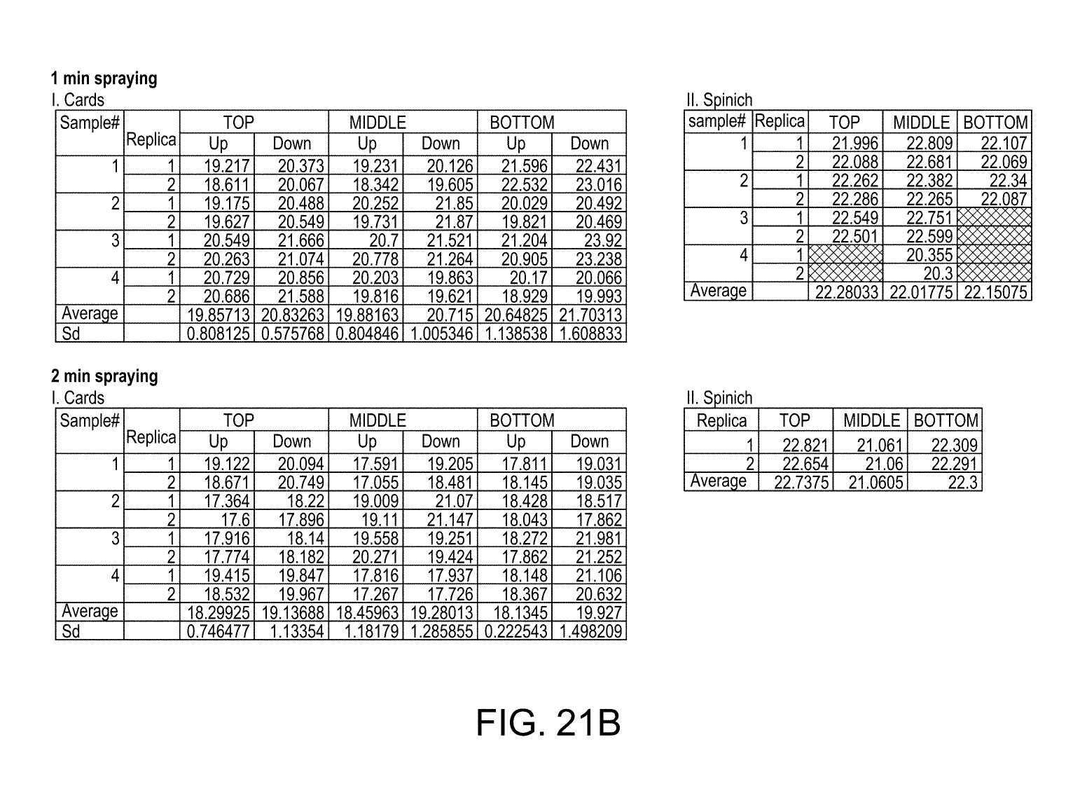

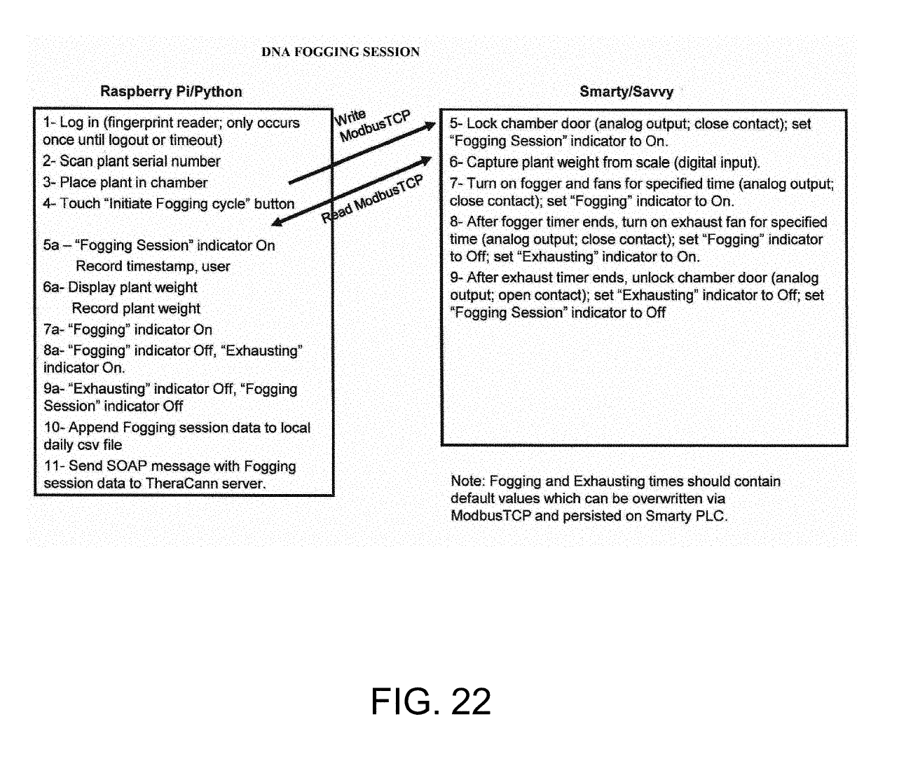

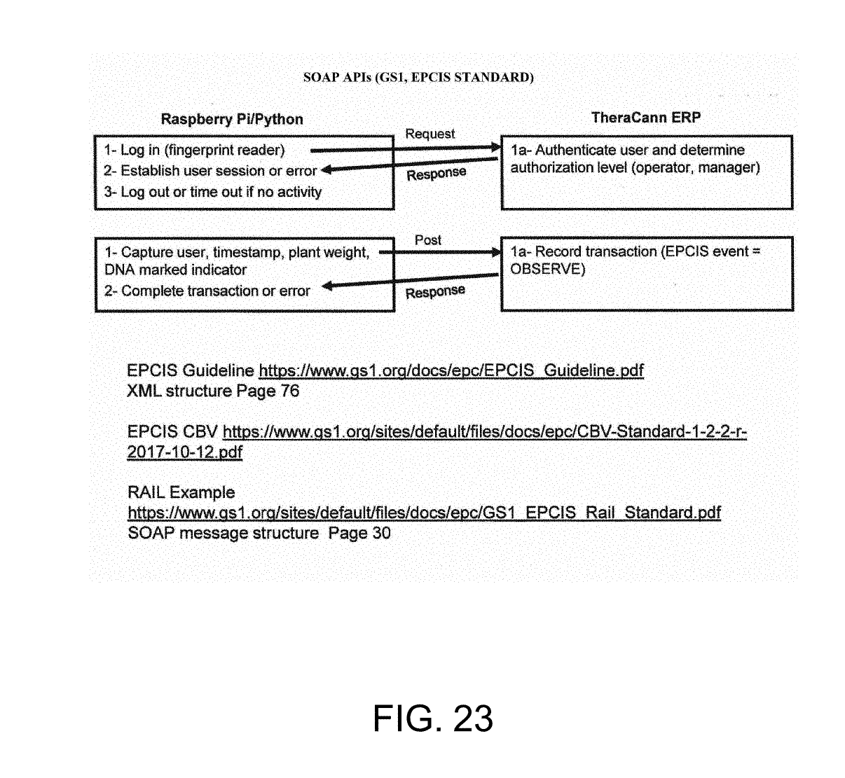

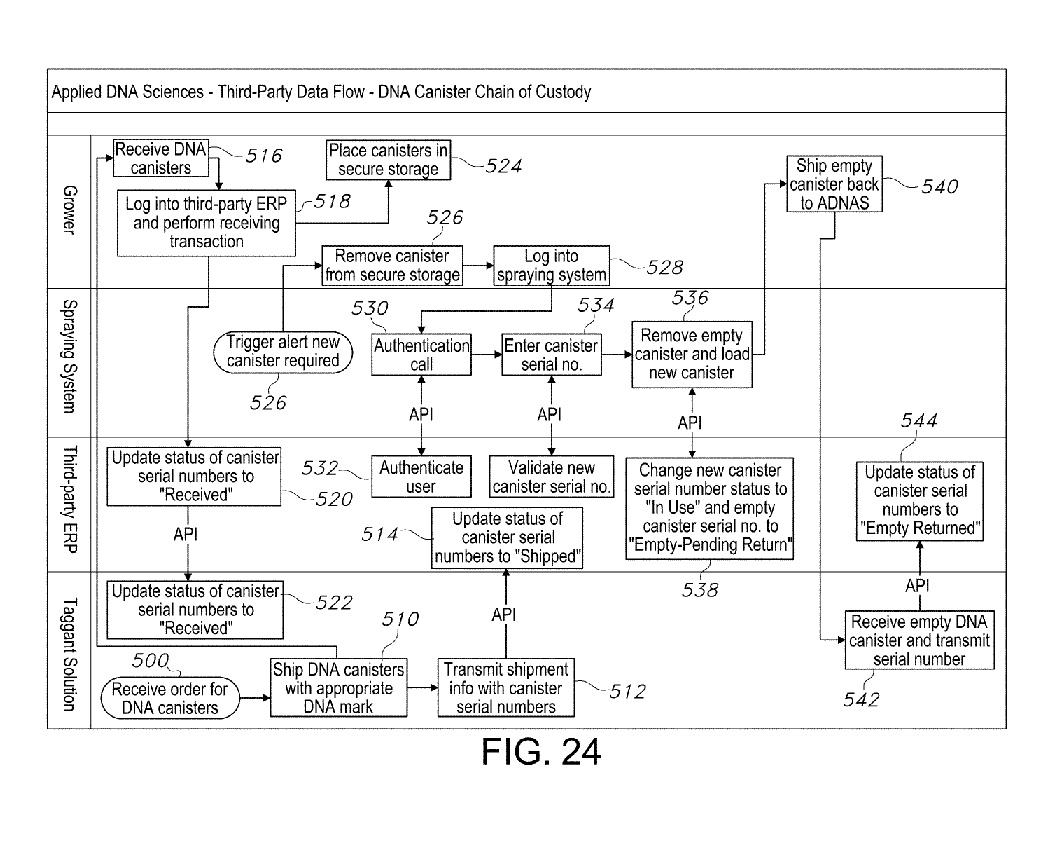

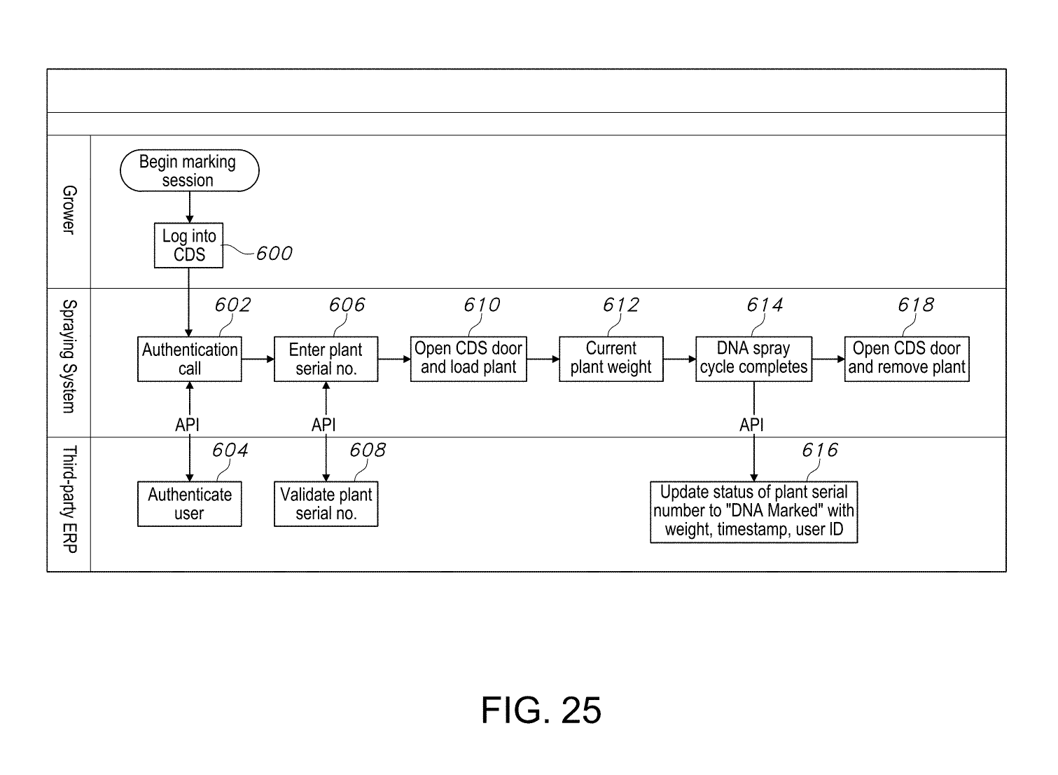

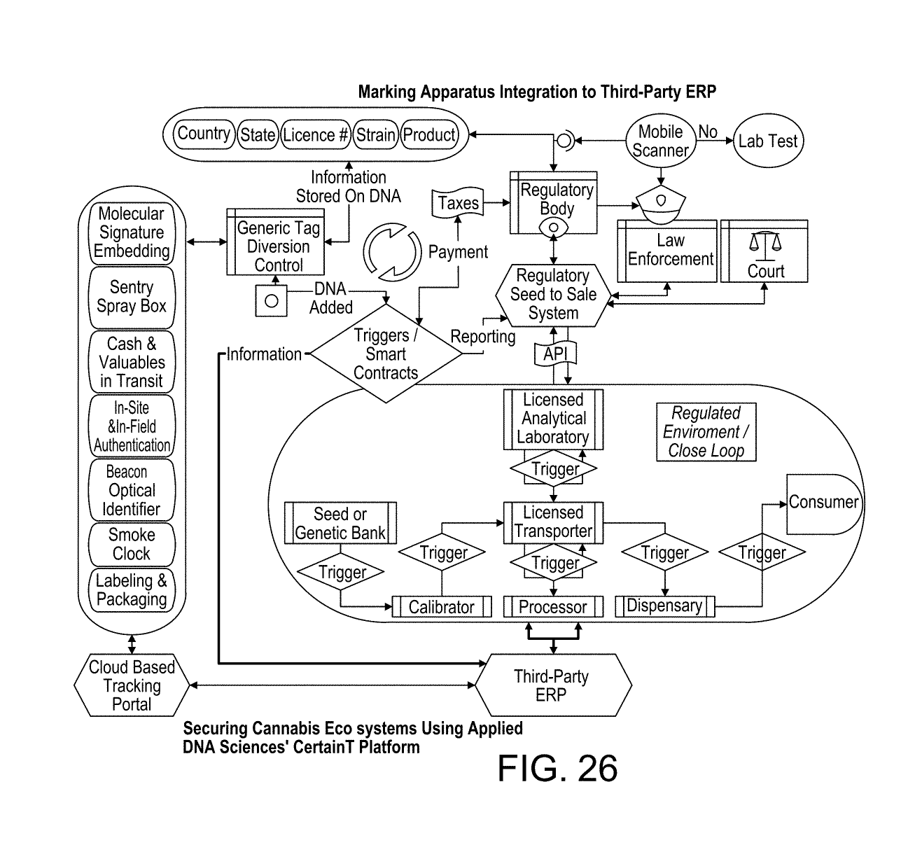

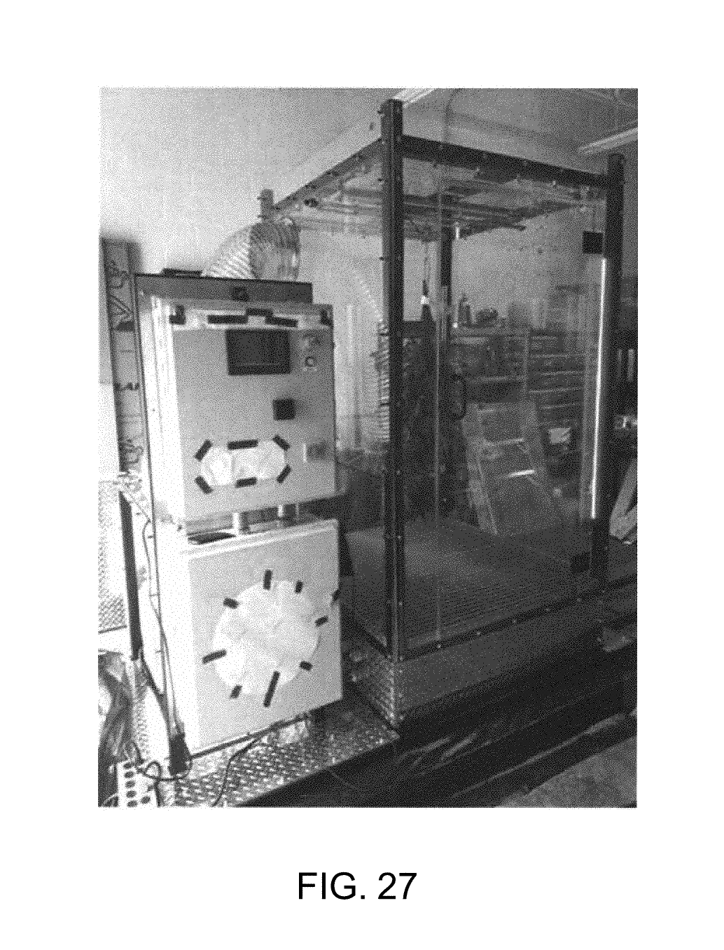

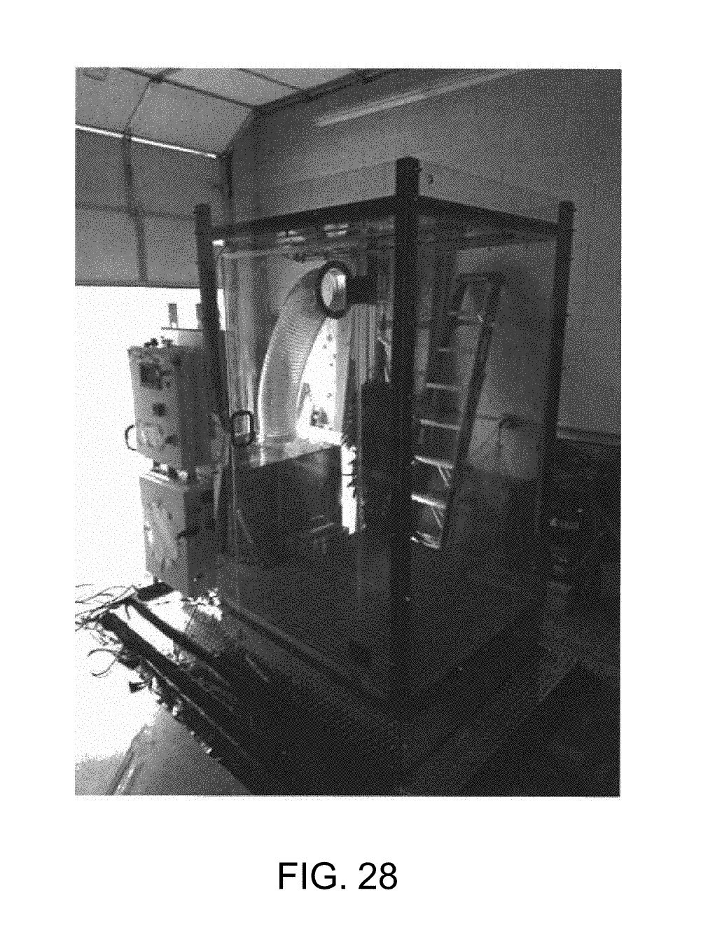

With reference to FIG. 21A, the test results of a fogging system test using spinach as the plant material are shown. A protein card was used as a surrogate of cannabis plant leaves, and positioned at level to the floor, therefore, there is up or bottom side of the card. Spinach leaves were hung inside the spraying chamber. Once all content was secured, the fogging system was run through a tagging cycle. DNA tags from the surface of the spinach was rinsed with 1xTE buffer, the rinsate was tested by the qPCR assay. Top, Middle or Bottom is where the cards/spinach were located inside the fogging chamber. Bottom is about half meter from the ground. Results were obtained with MyGo qPCR instrument and a TaqMan assay specific to the sequence of the DNA taggant sprayed. Results were presented as the Cq value from each sample. All samples showed robust and indelible tagging with the DNA taggant after once tagging cycle.

1. A system for tracking the origin of cannabis products and cannabis derivative products comprising:
a nucleic acid tag;
a marking apparatus; and
a nucleic acid tag authentication apparatus; and
optionally, a digital data repository wherein the marking apparatus and the nucleic acid tag authentication apparatus are configured to digitally communicate with the digital data repository system; and one or more remote user interfaces to the digital data repository.

2. The system of claim 1 wherein the nucleic acid tag is a DNA tag.

3. The system of claim 2 wherein the DNA tag comprises one or more amplicons and one or more oligonucleotides.

4. The system of claim 1 wherein the nucleic acid tag comprises a combinatorial DNA mark.

5. The system of claim 1 wherein the nucleic acid tag is created on demand via an oligonucleotide synthesis apparatus.

6. The system of claim 5 wherein the oligonucleotide synthesis apparatus is configured to digitally communicate with the digital data repository.

7. The system of claim 1 wherein the marking apparatus is selected from the group consisting of:
a misting system, electrostatic spray system, atomized spray system, pressurized spray system, sprinkler system, fogging system, cooling system, handheld spray device, and powder dusting system.

8. The system of claim 1 wherein the authentication apparatus is selected from the group consisting of: capillary electrophoresis apparatus, qPCR apparatus, PCR apparatus, microarray, microarray and microarray scanning apparatus, multiplexing apparatus, and next generation sequencing apparatus.

9. The system of claim 1 wherein the digital data repository is a continually reconciled distributed database blockchain.

10. A method of tracking the origin of cannabis and cannabis derivative products comprising:
applying a nucleic acid tag to a cannabis plant to create a nucleic acid tagged cannabis plant, said tag imparting at least one piece of information about the origin of the cannabis plant;
processing the nucleic acid tagged cannabis plant to create nucleic acid tagged cannabis products and/or nucleic acid tagged cannabis derivative products; and
interrogating a nucleic acid tagged cannabis product and/or cannabis derivative product to ascertain information from the nucleic acid tag; and
optionally, querying a digital data repository for information associated with the DNA tag information,
thereby ascertaining the origin of the cannabis product and/or cannabis derivative product

11. The method of claim 10 wherein the nucleic acid tag is a DNA tag.

12. The method of claim 10 wherein the nucleic acid tag comprises more than one different DNA fragments of known sequences

13. The method of claim 10 wherein the nucleic acid tag comprises a combinatorial DNA mark.

14. The method of claim 10 wherein the nucleic acid tag is created on demand via an oligonucleotide synthesis apparatus.

15. The method of claim 10 wherein the nucleic acid tag is applied to a cannabis plant via an apparatus selected from the group consisting of: a misting system, electrostatic spray system, atomized spray system, pressurized spray system, sprinkler system, fogging system, cooling system, handheld spray device, and powder dusting system.

16. The method of claim 10 wherein the nucleic acid tagged cannabis product and/or cannabis derivative product is interrogated via an apparatus selected from the group consisting of:
capillary electrophoresis apparatus, qPCR apparatus, PCR apparatus, microarray, microarray and microarray scanning apparatus, multiplexing apparatus, and next generation sequencing apparatus.

17. A system of applying a taggant to plant material comprising:
a spraying chamber including panels defining an interior for receiving plant material;
an attachment assembly adapted to secure the plant material within the spraying chamber;
a fogging unit in fluid communication with the spraying chamber;
an aqueous supply of taggant solution in fluid communication with the fogging unit, the fogging unit producing a mist from the solution without the use of heat;
a mist dispersal device disposed adjacent the fogging unit for disbursing mist produced by the fogging unit to coat the plant material with the nucleic acid tag solution; and
a processing device for controlling the operations of the fogging unit and mist dispersal device.

18. A DNA tag comprising an amplicon of a known DNA sequence imparting high-level data produced by PCR, and an oligonucleotide of a known DNA sequence imparting low-level data produced by oligonucleotide synthesis, wherein the amplicon of a known DNA sequence comprises more nucleotides than the oligonucleotide of a known DNA sequence.
